SEQUENCE LISTING (20 sequences)

SEQ ID NO: 1 - DNA, 1488 bp, Homo sapiens
atgccatgtgtgtggggtacgaccagtgggccaccagccagctcattgggaccattttcttctgtgtgggaatcactactttgctacagacaacgtttggatgcagatgtttcagttgccaatggaacagcagagctgttgcacacagaacacatctggtatccccggatccgagagatccagggggccatcatcatgtcctcactgatagaagtagtcatcggcctcctcggcctgcctggggctctactgaagtacatcggtcccttgaccattacacccacggtggccctaattggcctctctggtttccaggcagcgggggagagagccgggaagcactggggcattgccatgctgacaatattcctagtattactgttttctcaatacgccagaaatgttaaatttcctctcccgatttataaatccaagaaaggatggactgcgtacaagttacagctgttcaaaatgttccctatcatcctggccatcctggtatcctggctgctctgcttcatcttcacggtgacagatgtcttccctcccgacagcacaaagtatggcttctatgctcgcacagatgccaggcaaggcgtgcttctggtagccccgtggtttaaggttccatacccatttcagtggggactgcccaccgtgtctgcggccggtgtcatcggcatgctcagtgccgtggtcgccagcatcatcgagtctattggtgactactacgcctgtgcacggctgtcctgtgccccacccccccccatccacgcaataaacaggggaattttcgtggaaggcctctcctgtgttcttgatggcatatttggtactgggaatggctctacttcatccagtcccaacattggagttttgggaattacaaaggtcggcagccgccgcgtgatacagtgcggagcagccctcatgctcgctctgggcatgatcgggaagttcagcgccctctttgcgtcccttccggatcctgtgctgggagccctgttctgcacgctctttggaatgatcacagctgttggcctctctaacctgcagttcattgatttaaattcttcccggaacctctttgtgcttggattttcgatcttctttgggctcgtccttccaagttacctcagacagaaccctctggtcacagggataacaggaatcgatcaagtgttgaacgtccttctcacaactgctatgtttgtagggggctgtgtggcttttatcctggataacaccatcccaggcactccagaggaaagaggaatccggaaatggaagaagggtgtgggcaaagggaacaaatcactcgacggcatggagtcgtacaatttgccatttggcatgaacattataaaaaaatacagatgcttcagctacttacccatcagcccaacctttgtgggctacacatggaaaggcctcaggaagagcgacaacagccggagttcagatgaagactcccaggccacgggatag

SEQ ID NO: 2 - DNA, 1710 bp, Homo sapiens
atggcgcccaccctggccactgcccatcggcgccgctggtggatggcctgcacggccgtgctggagaacctcctcttctcggcagtcctcctgggctggggctcgctgctcatcatgctcaagtcagagggcttttactcctacctgtgtaccgagccagagaatgtcaccaatggcacagtgggcggcacagcagagccggggcacgaggaggtgagctggatgaacggctggctcagctgccaggcccaggacgagatgctaaatttggccttcactgtgggctcctttctgctcagtgccatcaccctgcccctgggtatcgtcatggacaagtatggcccgaggaagctcaggctgctgggcagcgcctgcttcgcggtttcctgcttgctgattgcgtacggagcaagtaaaccaaacgctctctccgtgctcatcttcatcgccctggctctgaatggctttggtgggatgtgtatgaccttcacctcattaacactgcccaacatgttcggcgaccttcggtccacgtttattgccttgatgattgggtcctacgcctcctcggcagtcacctttccaggaatcaagctcatctatgatgctggtgtctccttcatcgtcgtcctcgtggtctgggccggctgctccgggctggttttcctcaactgcttctttaactggccccttgagcccttcccggggccggaggacatggactactcggtgaagatcaagttcagctggctgggctttgaccacaagatcacagggaagcagttctacaagcaggtgaccacggtgggccggcgcctgagtgtgggcagctccatgaggagtgccaaggagcaggtggcgctgcaggagggccacaagctgtgcctgtccaccgtcgacctggaggtgaagtgccagccggatgccgcagtggccccctccttcatgcacagcgtgttcagccccatcctgctgctcagcctggtcaccatgtgcgtcacgcagctgcggctcatcttctacatgggggctatgaacaacatcctcaagttcctggtcagcggcgaccagaagacagttggcctctacacctccatcttcggcgtgctccagctgctgtgcctgctgacggcccccgtcattggctacatcatggactggaggctgaaggagtgtgaagacgcctccgaggagcccgaggagaaagacgccaaccaaggcgagaagaaaaagaagaagcgggaccggcagatccagaagatcactaatgccatgcgggccttcgccttcaccaacctgctgctcgtgggctttggggtgacctgcctcattcccaacctgcctctccagatcctctccttcatcctgcacacaatcgtgcgaggattcatccactccgctgtcgggggcctgtacgctgccgtgtacccctccacccagttcggcagcctcacgggactgcagtctctgatcagcgcgctcttcgcccttctgcagcagccgctgtttctggccatgatgggtcctctccagggagaccctctgtgggtgaacgtggggctgctccttctcagcctgctgggcttctgcctcccgctctacctgatctgctaccggcgccagctggagcggcagctgcagcagaggcaggaggatgacaaactcttcctcaaaatcaacggctcgtccaaccaggaggccttcgtgtag

SEQ ID NO: 3 - DNA, 1428 bp, Homo sapiens
atggccgttgtctcagaggacgactttcagcacagttcaaactccacctacagaaccacaagcagcagtctccgagctgaccaggaggcactgcttgagaagctgctggaccgcccgccccctggcctgcagaggcccgaggaccgcttctgtggcacatacatcatcttcttcagcctgggcattggcagtctactgccatggaacttctttatcactgccaaggagtactggatgttcaaactccgcaactcctccagcccagccaccggggaggaccctgagggctcagacatcctgaactactttgagagctaccttgccgttgcctccaccgtgccctccatgctgtgcctggtggccaacttcctgcttgtcaacagggttgcagtccacatccgtgtcctggcctcactgacggtcatcctggccatcttcatggtgataactgcactggtgaaggtggacacttcctcctggacccgtggcttttttgcggtcaccattgtctgcatggtgatcctcagcggtgcctccactgtcttcagcagcagcatctacggcatgaccggctcctttcctatgaggaactcccaggcactgatatcaggaggagccatgggcgggacggtcagcgccgtggcctcattggtggacttggctgcatccagtgatgtgaggaacagcgccctggccttcttcctgacggccactgtcttcctcgtgctctgcatgggactctacctgctgctgtccaggctggagtatgccaggtactacatgaggcctgttcttgcggcccatgtgttttctggtgaagaggagcttccccaggactccctcagtgccccttcggtggcctccagattcattgattcccacacaccccctctccgccccatcctgaagaagacggccagcctgggcttctgtgtcacctacgtcttcttcatcaccagcctcatctaccccgccatctgcaccaacatcgagtccctcaacaagggttcgggctcactgtggaccaccaagtttttcatccccctcactaccttcctcctgtacaactttgctgacctatgtggccggcagctcaccgcctggatccaggtgccagggcccaatagcaaggcgctcccagggttcgtgctcctccggacctgcctcatccccctcttcgtgctctgtaactaccagccccgcgtccacctgaagactgtggtcttccagtccgatgtgtaccccgcactcctcagctccctgctggggctcagcaacggctacctcagcaccctggccctcctctacgggcctaagattgtgcccagggagctggctgaggccacgggagtggtgatgtccttttatgtgtgcttgggcttaacactgggctcagcctgctctaccctcctggtgcacctcatctag

SEQ ID NO: 4 - DNA, 1095 bp, Homo sapiens
atgaatagggcccctctgaagcggtccaggatcctgcacatggcgctgaccggggcctcagacccctctgcagaggcagaggccaacggggagaagccctttctgctgcgggcattgcagatcgcgctggtggtctccctctactgggtcacctccatctccatggtgttccttaataagtacctgctggacagcccctccctgcggctggacacccccatcttcgtcaccttctaccagtgcctggtgaccacgctgctgtgcaaaggcctcagcgctctggccgcctgctgccctggtgccgtggacttccccagcttgcgcctggacctcagggtggcccgcagcgtcctgcccctgtcggtggtcttcatcggcatgatcaccttcaataacctctgcctcaagtacgtcggtgtggccttctacaatgtgggccgctcactcaccaccgtcttcaacgtgctgctctcctacctgctgctcaagcagaccacctccttctatgccctgctcacctgcggtatcatcatcgggggcttctggcttggtgtggaccaggagggggcagaaggcaccctgtcgtggctgggcaccgtcttcggcgtgctggctagcctctgtgtctcgctcaacgccatctacaccacgaaggtgctcccggcggtggacggcagcatctggcgcctgactttctacaacaacgtcaacgcctgcatcctcttcctgcccctgctcctgctgctcggggagcttcaggccctgcgtgactttgcccagctgggcagtgcccacttctgggggatgatgacgctgggcggcctgtttggctttgccatcggctacgtgacaggactgcagatcaagttcaccagtccgctgacccacaatgtgtcgggcacggccaaggcctgtgcccagacagtgctggccgtgctctactacgaggagaccaagagcttcctctggtggacgagcaacatgatggtgctgggcggctcctccgcctacacctgggtcaggggctgggagatgaagaagactccggaggagcccagccccaaagacagcgagaagagcgccatgggggtgtga

SEQ ID NO: 5 - DNA, 966 bp, Homo sapiens
atggcgacgggcggccagcagaaggagaacacgctgcttcacctcttcgccggcgggtgtggaggcacagttggtgctattttcacttgtccactagaagtcattaagacacggttgcagtcttcaagattagctctccggacagtctactatcctcaggttcatctggggaccattagtggagctggaatggtgagaccaacatccgtgacacctggactctttcaggttctgaagtcgatcttggagaaagagggaccaaagtcactttttagaggcttgggtccaaatttggttggagttgcaccatcaagggctgtatactttgcatgttactccaaagccaaagagcaatttaatggcattttcgtgcctaacagcaatattgtgcatattttctcagctggctctgcagcttttatcacaaattccttaatgaatcctatatggatggttaaaacccgaatgcagctagaacagaaagtgaggggctctaagcagatgaatacactccagtgtgctcgttacgtttaccagaccgaaggcattcgtggcttctatagaggattaactgcctcgtatgctggaatttccgaaactataatctgctttgctatttatgaaagtttaaagaagtatctgaaagaagctccattagcctcttctgcaaatgggactgagaaaaattccacaagtttttttggacttatggcagctgctgctctttctaagggctgtgcctcctgcattgcttatccacacgaagtcataaggacgaggctccgggaagagggcaccaagtacaagtcttttgtccagacggcgcgcctggtgttccgggaagaaggctaccttgccttttatagaggactgtttgcccagcttatccggcagatcccaaatactgccattgtgttgtctacttatgagttaattgtgtacctgttagaagaccgtactcagtaa

SEQ ID NO: 6 - DNA, 2061 bp, Homo sapiens
atggccatagaccggcggcgcgaggcggcgggcggcgggcctgggcggcagccggccccggccgaggagaacggctccctgccgcccggggacgcggcggcctcggcgcccctcgggggacgcgcgggccccggcggcggcgcggagatccagccgctgcccccactgcatcctggaggcggcccgcacccgagctgctgctccgcggctgcggccccgagcctcttgttgctggactatgacgggtcggtgctgcccttcctcgggggcctgggcgggggctatcagaagaccctcgtgctgctcacctggatcccggcgctgttcatcggcttcagccagttctcggactcgttcctcctggaccagcccaacttctggtgccgcggggccggcaaaggcaccgagctggcaggggtcaccaccacaggccggggcggggacatgggcaactggaccagcctccccaccacccccttcgccactgccccctgggaggctgcgggcaaccggagcaacagcagcggcgcggacggaggcgacacaccacccctgccatcccctccggacaagggggacaacgcctccaactgtgactgccgcgcatgggactacggcatccgcgccggcctcgtccagaacgtggtcagcaagtgggatcttgtgtgtgataatgcctggaaggtccatatcgctaagttctccttactggttggattaatctttggctacctaataactggatgcattgctgactgggtcggccggcggcctgtgctgctgttttccatcatcttcattctgatctttggactgactgtggcactgtcagtgaatgtgacaatgttcagcacactcaggttctttgaaggattttgcctggctggaatcattctcaccttgtatgctttacgaatagagctgtgcccccctggaaaacggttcatgattacgatggtggcgagcttcgtggccatggcgggccagttcctcatgcctgggctagccgccctgtgccgggattggcaggtgctgcaggccctcatcatctgccccttcctgctcatgctgctctactggtcgatattccccgagtccctccggtggctaatggccacccagcagtttgagtctgcaaagaggctgatcctccacttcacacagaagaatcgcatgaaccctgagggcgacatcaagggtgtgataccagagctggagaaagagctttcccggaggcccaagaaggtctgcatcgtgaaggtggtggggacacggaacctgtggaagaacattgtggtcctgtgtgtgaactcgctgacggggtacgggatccaccactgctttgccaggagcatgatgggccacgaggtgaaggtgccgctcctggagaacttctatgctgactactataccacggccagcatcgcgctggtgtcctgcctggccatgtgcgtggtggtccgattcctcgggcgcaggggagggctgctgctcttcatgatcctcaccgccctggcctcactcctgcagctcggcctcctcaacctgattggaaagtacagccagcacccagactcagggatgagtgacagcgtcaaggacaaattttccatcgcgttttccatcgtgggcatgtttgcctcccatgcggtggggagcctcagcgtgttcttctgtgcggagatcaccccgacggtgataaggtgtggcgggctggggctggtgctggccagcgcgggcttcggcatgctgacggcacccatcatcgagctgcacaaccagaaaggctacttcctgcaccacatcatctttgcctgctgcacgctcatctgcatcatctgcatcctcctgctgcccgagagcagggaccagaacctgcctgagaacatttctaacggggagcactacacgcgccagccgctgctgccgcacaagaagggggagcagccactgctgctcaccaacgccgagctcaaggactactcgggcctccacgatgccgcagccgcgggtgacacactgcccgagggtgccacggccaacggcatgaaggccatgtag

SEQ ID NO: 7 - DNA, 270 bp, Homo sapiens
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaa

SEQ ID NO: 8 - DNA, 285 bp, Homo sapiens
atggccccactgaagatgctggccctggtcaccctcctcctgggggcttctctgcagcacatccacgcagctcgagggaccaatgtgggccgggagtgctgcctggagtacttcaagggagccattccccttagaaagctgaagacgtggtaccagacatctgaggactgctccagggatgccatcgtttttgtaactgtgcagggcagggccatctgttcggaccccaacaacaagagagtgaagaatgcagttaaatacctgcaaagccttgagaggtcttga

SEQ ID NO: 9 - DNA, 510 bp, Homo sapiens
atggccccgccctgggtgcccgccatgggcttcacgctggcgcccagcctggggtgcttcgtgggctcccgctttgtccacggcgagggtctccgctggtacgccggcctgcagaagccctcgtggcacccgccccactgggtgctgggccctgtctggggcacgctctactcagccatggggtacggctcctacctggtctggaaagagctgggaggcttcacagagaaggctgtggttcccctgggcctctacactgggcagctggccctgaactgggcatggccccccatcttctttggtgcccgacaaatgggctgggccttggtggatctcctgctggtcagtggggcggcggcagccactaccgtggcctggtaccaggtgagcccgctggccgcccgcctgctctacccctacctggcctggctggccttcacgaccacactcaactactgcgtatggcgggacaaccatggctggcgtgggggacggcggctgccagagtga

SEQ ID NO: 10 - DNA, 1122 bp, Homo sapiens
atgaccgaggtgctgtggccggctgtccccaacgggacggacgctgccttcctggccggtccgggttcgtcctgggggaacagcacggtcgcctccactgccgccgtctcctcgtcgttcaaatgcgccttgaccaagacgggcttccagttttactacctgccggctgtctacatcttggtattcatcatcggcttcctgggcaacagcgtggccatctggatgttcgtcttccacatgaagccctggagcggcatctccgtgtacatgttcaatttggctctggccgacttcttgtacgtgctgactctgccagccctgatcttctactacttcaataaaacagactggatcttcggggatgccatgtgtaaactgcagaggttcatctttcatgtgaacctctatggcagcatcttgtttctgacatgcatcagtgcccaccggtacagcggtgtggtgtaccccctcaagtccctgggccggctcaaaaagaagaatgcgatctgtatcagcgtgctggtgtggctcattgtggtggtggcgatctcccccatcctcttctactcaggtaccggggtccgcaaaaacaaaaccatcacctgttacgacaccacctcagacgagtacctgcgaagttatttcatctacagcatgtgcacgaccgtggccatgttctgtgtccccttggtgctgattctgggctgttacggattaattgtgagagctttgatttacaaagatctggacaactctcctctgaggagaaaatcgatttacctggtaatcattgtactgactgtttttgctgtgtcttacatccctttccatgtgatgaaaacgatgaacttgagggcccggcttgattttcagaccccagcaatgtgtgctttcaatgacagggtttatgccacgtatcaggtgacaagaggtctagcaagtctcaacagttgtgtggaccccattctctatttcttggcgggagatactttcagaaggagactctcccgagccacaaggaaagcttctagaagaagtgaggcaaatttgcaatccaagagtgaagacatgaccctcaatattttacctgagttcaagcagaatggagatacaagcctgtga

SEQ ID NO: 11 - DNA, 1113 bp, Homo sapiens
atgctgggggcagtggaaggccccaggtggaagcaggcggaggacattagagacatctacgacttccgagatgttctgggcacgggggccttctcggaggtgatcctggcagaagataagaggacgcagaagctggtggccatcaaatgcattgccaaggaggccctggagggcaaggaaggcagcatggagaatgagattgctgtcctgcacaagatcaagcaccccaacattgtagccctggatgacatctatgagagtgggggccacctctacctcatcatgcagctggtgtcgggtggggagctctttgaccgtattgtggaaaaaggcttctacacggagcgggacgccagccgcctcatcttccaggtgctggatgctgtgaaatacctgcatgacctgggcattgtacaccgggatctcaagccagagaatctgctgtactacagcctggatgaagactccaaaatcatgatctccgactttggcctctccaagatggaggacccgggcagtgtgctctccaccgcctgtggaactccgggatacgtggcccctgaagtcctggcccagaagccctacagcaaggctgtggattgctggtccataggtgtcatcgcctacatcttgctctgcggttaccctcccttctatgacgagaatgatgccaaactctttgaacagattttgaaggccgagtacgagtttgactctccttactgggacgacatctctgactctgccaaagatttcatccggcacttgatggagaaggacccagagaaaagattcacctgtgagcaggccttgcagcacccatggattgcaggagatacagctctagataagaatatccaccagtcggtgagtgagcagatcaagaagaactttgccaagagcaagtggaagcaagccttcaatgccacggctgtggtgcggcacatgaggaaactgcagctgggcaccagccaggaggggcaggggcagacggcgagccatggggagctgctgacaccagtggctggggggccggcagctggctgttgctgtcgagactgctgcgtggagccgggcacagaactgtcccccacactgccccaccagctctag

SEQ ID NO: 12 - DNA, 2343 bp, Homo sapiens
atggcgccctacagcctactggtgactcggctgcagaaagctctgggtgtgcggcagtaccatgtggcctcagtcctgtgccaacgggccaaggtggcgatgagccactttgagcccaacgagtacatccattatgacctgctagagaagaacattaacattgttcgcaaacgactgaaccggccgctgacactctcggagaagattgtgtatggacacctggatgaccccgccagccaggaaattgagcgaggcaagtcgtacctgcggctgcggccggaccgtgtggccatgcaggatgcgacggcccagatggccatgctccagttcatcagcagcgggctgtccaaggtggctgtgccatccaccatccactgtgaccatctgattgaagcccaggttgggggcgagaaagacctgcgccgggccaaggacatcaaccaggaagtttataatttcctggcaactgcaggtgccaaatatggcgtgggcttctggaagcctggatctggaatcattcaccagattattctggaaaactatgcgtaccctggtgttcttctgattggcactgactcccacacccccaatggtggcggccttgggggcatctgcattggagttgggggtgccgatgctgtggatgtcatggctgggatcccctgggagctgaagtgccccaaggtgattggcgtgaagctgacgggctctctctccggttggtcctcacccaaagatgtgatcctgaaggtggcaggcatcctcacggtgaaaggtggcacaggtgcaatcgtggaataccacgggcctggtgtagactccatctcctgcactggcatggcgacaatctgcaacatgggtgcagaaattggggccaccacttccgtgttcccttacaaccacaggatgaagaagtacctgagcaagaccggccgggaagacattgccaatctagctgatgaattcaaggatcacttggtgcctgaccctggctgccattatgaccaactaattgaaattaacctcagtgagctgaagccacacatcaatgggcccttcacccctgacctggctcaccctgtggcagaagtgggcaaggtggcagagaaggaaggatggcctctggacatccgagtgggtctaattggtagctgcaccaattcaagctatgaagatatggggcgctcagcagctgtggccaagcaggcactggcccatggcctcaagtgcaagtcccagttcaccatcactccaggttccgagcagatccgcgccaccattgagcgggacggctatgcacagatcttgagggatctgggtggcattgtcctggccaatgcttgtggcccctgcattggccagtgggacaggaaggacatcaagaagggggagaagaacacaatcgtcacctcctacaacaggaacttcacgggccgcaacgacgcaaaccccgagacccatgcctttgtcacgtccccagagattgtcacagccctggccattgcgggaaccctcaagttcaacccagagaccgactacctgacgggcacggatggcaagaagttcaggctggaggctccggatgcagatgagcttcccaaaggggagtttgacccagggcaggacacctaccagcacccacccaaggacagcagcgggcagcatgtggacgtgagccccaccagccagcgcctgcagctcctggagccttttgacaagtgggatggcaaggacctggaggacctgcagatcctcatcaaggtcaaagggaagtgtaccactgaccacatctcagctgctggcccctggctcaagttccgtgggcacttggataacatctccaacaacctgctcattggtgccatcaacattgaaaacggcaaggccaactccgtgcgcaatgccgtcactcaggagtttggccccgtccctgacactgcccgctactacaagaaacatggcatcaggtgggtggtgatcggagacgagaactacggcgagggctcgagccgggagcatgcagctctggagcctcgccaccttgggggccgggccatcatcaccaagagctttgccaggatccacgagaccaacctgaagaaacagggcctgctgcctctgaccttcgctgacccggctgactacaacaagattcaccctgtggacaagctgaccattcagggcctgaaggacttcacccctggcaagcccctgaagtgcatcatcaagcaccccaacgggacccaggagaccatcctcctgaaccacaccttcaacgagacgcagattgagtggttccgcgctggcagtgccctcaacagaatgaaggaactgcaacagtga

SEQ ID NO: 13 - DNA, 4503 bp, Homo sapiens
atggatccaagtatgggtgtgaattctgttaccatttctgttgagggtatgacttgcaattcctgtgtttggaccattgagcagcagattggaaaagtgaatggtgtgcatcacattaaggtatcactggaagaaaaaaatgcaactattatttatgaccctaaactacagactccaaagaccctacaggaagctattgatgacatgggctttgatgctgttatccataatcctgaccctctccctgttttaactgacaccttgtttctgactgttacggcgtcactgactttgccatgggaccatatccaaagcacattgctgaagaccaagggtgtgacagacattaaaatttaccctcagaaaagaactgtagcagtgacaataatcccttctatagtgaatgccaatcagataaaagagctggttccagaactcagtttagatactgggacactggagaaaaagtcaggagcttgtgaagatcatagtatggctcaagctggtgaagtcgtgctgaagatgaaagtggaagggatgacctgccattcatgtactagcactattgaaggaaaaattgggaaactgcaaggtgttcagcgaattaaagtctccctggacaatcaagaagctactattgtttatcaacctcatcttatctcagtagaggaaatgaaaaagcagattgaagctatgggctttccagcatttgtcaaaaagcagcccaagtacctcaaattgggagctattgatgtagaacgtctaaagaacacaccagttaaatcctcagaagggtcacagcaaaggagtccatcatataccaatgattcaacagccactttcatcattgatggcatgcattgtaaatcatgtgtgtcaaatattgaaagtactttatctgcactccaatatgtaagcagcatagtagtttctttagagaataggtctgccattgtgaagtataatgcaagctcagtcactccagaatccctgagaaaagcaatagaggctgtatcaccggggctatatagagttagtatcacaagtgaagttgagagtacctcaaactctccctccagctcatctcttcagaagattcctttgaatgtagttagccagcctctgacacaagaaactgtgataaacattgatggcatgacttgtaattcctgtgtgcagtctattgagggtgtcatatcaaaaaagccaggtgtaaaatccatacgagtctcccttgcaaatagcaatgggactgttgagtatgatcctctactaacctctccagaaacgttgagaggagcaatagaagacatgggatttgatgctaccttgtcagacacgaatgagccgttggtagtaatagctcagccttcatcggaaatgccgcttttgacttcaactaatgaattttatactaaagggatgacaccagttcaagacaaggaggaaggaaagaattcatctaagtgttacatacaggtcactggcatgacttgcgcttcctgtgtagcaaacattgaacggaatttaaggcgggaagaaggaatatattctatacttgtggccctgatggctggcaaggcagaagtaaggtataatcctgctgttatacaacccccaatgatagcagagttcatccgagaacttggatttggagccactgtgatagaaaatgctgatgaaggagatggtgttttggaacttgttgtgaggggaatgacgtgtgcctcctgcgtacataaaatagagtctagtctcacaaaacacagagggatcctatactgctccgtggccctggcaaccaacaaagcacatattaaatatgacccagaaattattggtcctagagatattatccatacaattgaaagcttaggttttgaagcttctttggtcaagaaggatcggtcagcaagtcacttagatcataaacgagaaataagacaatggagacggtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctcttccttaaactttacaggaaaccaacttacgagagttatgaactgcctgcccggagccagataggacagaagagtccttcagaaatcagcgttcatgttggaatagatgatacctcaaggaattctcctaaactgggtttgctggaccggattgttaattatagcagagcctctataaactcactactgtctgataaacgctccctaaacagtgttgttaccagtgaacctgacaagcactcactcctggtgggagacttcagggaagatgatgacactgcattataa

SEQ ID NO: 14 - DNA, 3720 bp, Homo sapiens
atgagccgcctgctctggaggaaggtggccggcgccaccgtcgggccagggccggttccagctccggggcgctgggtctccagctccgtccccgcgtccgaccccagcgacgggcagcggcggcggcagcagcagcagcagcagcagcagcagcagcaacagcagcctcagcagccgcaagtgctatcctcggagggcgggcagctgcggcacaacccattggacatccagatgctctcgagagggctgcacgagcaaatcttcgggcaaggaggggagatgcctggcgaggccgcggtgcgccgcagcgtcgagcacctgcagaagcacgggctctgggggcagccagccgtgcccttgcccgacgtggagctgcgcctgccgcccctctacggggacaacctggaccagcacttccgcctcctggcccagaagcagagcctgccctacctggaggcggccaacttgctgttgcaggcccagctgcccccgaagcccccggcttgggcctgggcggagggctggacccggtacggccccgagggggaggccgtacccgtggccatccccgaggagcgggccctggtgttcgacgtggaggtctgcttggcagagggaacttgccccacattggcggtggccatatccccctcggcctggtattcctggtgcagccagcggctggtggaagagcgttactcttggaccagccagctgtcgccggctgacctcatccccctggaggtccctactggtgccagcagccccacccagagagactggcaggagcagttagtggtggggcacaatgtttcctttgaccgagctcatatcagggagcagtacctgatccagggttcccgcatgcgtttcctggacaccatgagcatgcacatggccatctcagggctaagcagcttccagcgcagtctgtggatagcagccaagcagggcaaacacaaggtccagccccccacaaagcaaggccagaagtcccagaggaaagccagaagaggcccagcgatctcatcctgggactggctggacatcagcagtgtcaacagtctggcagaggtgcacagactttatgtaggggggcctcccttagagaaggagcctcgagaactgtttgtgaagggcaccatgaaggacattcgtgagaacttccaggacctgatgcagtactgtgcccaggacgtgtgggccacccatgaggttttccagcagcagctaccgctcttcttggagaggtgtccccacccagtgactctggccggcatgctggagatgggtgtctcctacctgcctgtcaaccagaactgggagcgttacctggcagaggcacagggcacttatgaggagctccagcgggagatgaagaagtcgttgatggatctggccaatgatgcctgccagctgctctcaggagagaggtacaaagaagacccctggctctgggacctggagtgggacctgcaagaatttaagcagaagaaagctaagaaggtgaagaaggaaccagccacagccagcaagttgcccatcgagggggctggggcccctggtgatcccatggatcaggaagacctcggcccctgcagtgaggaggaggagtttcaacaagatgtcatggcccgcgcctgcttgcagaagctgaaggggaccacagagctcctgcccaagcggccccagcaccttcctggacaccctggatggtaccggaagctctgcccccggctagacgaccctgcatggaccccgggccccagcctcctcagcctgcagatgcgggtcacacctaaactcatggcacttacctgggatggcttccctctgcactactcagagcgtcatggctggggctacttggtgcctgggcggcgggacaacctggccaagctgccgacaggtaccaccctggagtcagctggggtggtctgcccctacagagccatcgagtccctgtacaggaagcactgtctcgaacaggggaagcagcagctgatgccccaggaggccggcctggcggaggagttcctgctcactgacaatagtgccatatggcaaacggtagaagaactggattacttagaagtggaggctgaggccaagatggagaacttgcgagctgcagtgccaggtcaacccctagctctgactgcccgtggtggccccaaggacacccagcccagctatcaccatggcaatggaccttacaacgacgtggacatccctggctgctggtttttcaagctgcctcacaaggatggtaatagctgtaatgtgggaagcccctttgccaaggacttcctgcccaagatggaggatggcaccctgcaggctggcccaggaggtgccagtgggccccgtgctctggaaatcaacaaaatgatttctttctggaggaacgcccataaacgtatcagctcccagatggtggtgtggctgcccaggtcagctctgccccgtgctgtgatcaggcaccccgactatgatgaggaaggcctctatggggccatcctgccccaagtggtgactgccggcaccatcactcgccgggctgtggagcccacatggctcaccgccagcaatgcccggcctgaccgagtaggcagtgagttgaaagccatggtgcaggccccacctggctacacccttgtgggtgctgatgtggactcccaagagctgtggattgcagctgtgcttggagacgcccactttgccggcatgcatggctgcacagcctttgggtggatgacactgcagggcaggaagagcaggggcactgatctacacagtaagacagccactactgtgggcatcagccgtgagcatgccaaaatcttcaactacggccgcatctatggtgctgggcagccctttgctgagcgcttactaatgcagtttaaccaccggctcacacagcaggaggcagctgagaaggcccagcagatgtacgctgccaccaagggcctccgctggtatcggctgtcggatgagggcgagtggctggtgagggagttgaacctcccagtggacaggactgagggtggctggatttccctgcaggatctgcgcaaggtccagagagaaactgcaaggaagtcacagtggaagaagtgggaggtggttgctgaacgggcatggaaggggggcacagagtcagaaatgttcaataagcttgagagcattgctacgtctgacataccacgtaccccggtgctgggctgctgcatcagccgagccctggagccctcggctgtccaggaagagtttatgaccagccgtgtgaattgggtggtacagagctctgctgttgactacttacacctcatgcttgtggccatgaagtggctgtttgaagagtttgccatagatgggcgcttctgcatcagcatccatgacgaggttcgctacctggtgcgggaggaggaccgctaccgcgctgccctggccttgcagatcaccaacctcttgaccaggtgcatgtttgcctacaagctgggtctgaatgacttgccccagtcagtcgcctttttcagtgcagtcgatattgaccggtgcctcaggaaggaagtgaccatggattgtaaaaccccttccaacccaactgggatggaaaggagatacgggattccccagggtgaagcgctggatatttaccagataattgaactcaccaaaggctccttggaaaaacgaagccagcctggaccatag

SEQ ID NO: 15 - DNA, 1182 bp, Homo sapiens
atggcgctgaaggtagcgaccgtcgccggcagcgccgcgaaggcggtgctcgggccagcccttctctgccgtccctgggaggttctaggcgcccacgaggtcccctcgaggaacatcttttcagaacaaacaattcctccgtccgctaagtatggcgggcggcacacggtgaccatgatcccaggggatggcatcgggccagagctcatgctgcatgtcaagtccgtcttcaggcacgcatgtgtaccagtggactttgaagaggtgcacgtgagttccaatgctgatgaagaggacattcgcaatgccatcatggccatccgccggaaccgcgtggccctgaagggcaacatcgaaaccaaccataacctgccaccgtcgcacaaatctcgaaacaacatccttcgcaccagcctggacctctatgccaacgtcatccactgtaagagccttccaggcgtggtgacccggcacaaggacatagacatcctcattgtccgggagaacacagagggcgagtacagcagcctggagcatgagagtgtggcgggagtggtggagagcctgaagatcatcaccaaggccaagtccctgcgcattgccgagtatgccttcaagctggcgcaggagagcgggcgcaagaaagtgacggccgtgcacaaggccaacatcatgaaactgggcgatgggcttttcctccagtgctgcagggaggtggcagcccgctaccctcagatcaccttcgagaacatgattgtggataacaccaccatgcagctggtgtcccggccccagcagtttgatgtcatggtgatgcccaatctctatggcaacatcgtcaacaatgtctgcgcgggactggtcgggggcccaggccttgtggctggggccaactatggccatgtgtacgcggtgtttgaaacagctacgaggaacaccggcaagagtatcgccaataagaacatcgccaaccccacggccaccctgctggccagctgcatgatgctggaccacctcaagctgcactcctatgccacctccatccgtaaggctgtcctggcatccatggacaatgagaatatgcacactccggacatcgggggccagggcacaacatctgaagccatccaggacgtcatccgccacatccgcgtcatcaacggccgggccgtggaggcctag

SEQ ID NO: 16 - DNA, 930 bp, Homo sapiens
atggttgggttcaaggccacagatgtgccccctactgccactgtgaagtttcttggggctggcacagctgcctgcatcgcagatctcatcacctttcctctggatactgctaaagtccggttacagatccaaggagaaagtcaggggccagtgcgcgctacagccagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaagggacctctcccaatgttgctcgtaatgccattgtcaactgtgctgagctggtgacctatgacctcatcaaggatgccctcctgaaagccaacctcatgacagatgacctcccttgccacttcacttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggttcatgccctcctttctccgcttgggttcctggaacgtggtgatgttcgtcacctatgagcagctgaaacgagccctcatggctgcctgcacttcccgagaggctcccttctga

SEQ ID NO: 17 - DNA, 1338 bp, Homo sapiens
atggcggtagcgatcgctgcagcgagggtctggcggctaaaccgtggtttgagccaggctgccctcctgctgctgcggcagcctggggctcggggactggctagatctcacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaaggtgatggaggcctttgagcaggccgagcggaagcccaaacccaaccccaacctactcttctcagacgtgtatcaggagatgcccgcccagctccgcaagcagcaggagtctctggcccgccacctgcagacctacggggagcactacccactggatcacttcgataagtga

SEQ ID NO: 18 - DNA, 1962 bp, Homo sapiens
atgcggctccctgacctgagaccctggacctccctgctgctggtggacgcggctttactgtggctgcttcagggccctctggggactttgcttcctcaagggctgccaggactatggctggaggggaccctgcggctgggagggctgtgggggctgctaaagctaagagggctgctgggatttgtggggacactgctgctcccgctctgtctggccacccccctgactgtctccctgagagccctggtcgcgggggcctcacgtgctcccccagccagagtcgcttcagccccttggagctggctgctggtggggtacggggctgcggggctcagctggtcactgtgggctgttctgagccctcctggagcccaggagaaggagcaggaccaggtgaacaacaaagtcttgatgtggaggctgctgaagctctccaggccggacctgcctctcctcgttgccgccttcttcttccttgtccttgctgttttgggtgagacattaatccctcactattctggtcgtgtgattgacatcctgggaggtgattttgacccccatgcctttgccagtgccatcttcttcatgtgcctcttctcctttggcagctcactgtctgcaggctgccgaggaggctgcttcacctacaccatgtctcgaatcaacttgcggatccgggagcagcttttctcctccctgctgcgccaggacctcggtttcttccaggagactaagacaggggagctgaactcacggctgagctcggataccaccctgatgagtaactggcttcctttaaatgccaatgtgctcttgcgaagcctggtgaaagtggtggggctgtatggcttcatgctcagcatatcgcctcgactcaccctcctttctctgctgcacatgcccttcacaatagcagcggagaaggtgtacaacacccgccatcaggaagtgcttcgggagatccaggatgcagtggccagggcggggcaggtggtgcgggaagccgttggagggctgcagaccgttcgcagttttggggccgaggagcatgaagtctgtcgctataaagaggcccttgaacaatgtcggcagctgtattggcggagagacctggaacgcgccttgtacctgctcgtaaggagggtgctgcacttgggggtgcagatgctgatgctgagctgtgggctgcagcagatgcaggatggggagctcacccagggcagcctgctttcctttatgatctaccaggagagcgtggggagctatgtgcagaccctggtatacatatatggggatatgctcagcaacgtgggagctgcagagaaggttttctcctacatggaccgacagccaaatctgccttcacctggcacgcttgcccccaccactctgcagggggttgtgaaattccaagacgtctcctttgcatatcccaatcgccctgacaggcctgtgctcaaggggctgacgtttaccctacgtcctggtgaggtgacggcgctggtgggacccaatgggtctgggaagagcacagtggctgccctgctgcagaatctgtaccagcccacagggggacaggtgctgctggatgaaaagcccatctcacagtatgaacactgctacctgcacagccaggtggtttcagttgggcaggagcctgtgctgttctccggttctgtgaggaacaacattgcttatgggctgcagagctgcgaagatgataaggtgatggcggctgcccaggctgcccacgcagatgacttcatccaggaaatggagcatggaatatacacagatgtaggggagaagggaagccagctggctgcgggacagaaacaacgtctggccattgcccgggcccttgtacgagacccgcgggtcctcatcctggatgaggctactagtgccctagatgtgcagtgcgagcaggccaaaaccctttggaagttcatgatattttga

SEQ ID NO: 19 - DNA, 1425 bp, Homo sapiens
atggcggcccgggcgggtttccagtctgtggctccaagcggcggcgccggagcctcaggaggggcgggcgcggctgctgccttgggcccgggcggaactccggggcctcctgtgcgaatgggcccggctccgggtcaagggctgtaccgctccccgatgcccggagcggcctatccgagaccaggtatgttgccaggcagccgaatgacacctcagggaccttccatgggaccccctggctatggggggaacccttcagtccgacctggcctggcccagtcagggatggatcagtcccgcaagagacctgcccctcagcagatccagcaggtccagcagcaggcggtccaaaatcgaaaccacaatgcaaagaaaaagaagatggctgacaaaattctacctcaaaggattcgtgaactggtaccagaatcccaggcctatatggatctcttggcttttgaaaggaaactggaccagactatcatgaggaaacggctagatatccaagaggccttgaaacgtcccatcaagcaaaaacggaagctgcgaattttcatttctaacactttcaatccggctaagtcagatgccgaggatggggaagggacggtggcttcctgggagcttcgggtagaaggacggctcctggaggattcagccttgtccaaatatgatgccactaaacaaaagaggaagttctcttccttttttaagtccttggtgattgaactggacaaagacctgtatgggccagacaaccatctggtagaatggcacaggaccgccactacccaggagaccgatggctttcaggtgaagcggccgggagacgtgaatgtacggtgtactgtcctactgatgctggattaccagcctccccagtttaaattagacccccgcctagctcgactcctgggcatccatacccagactcgtccagtgatcatccaagcactgtggcaatatattaagacacataagctccaggaccctcacgagcgggagtttgtcatctgtgacaagtacctgcagcagatctttgagtctcaacgtatgaagttttcagagatccctcagcggctccatgccttgcttatgccaccagaacctatcatcattaatcatgtcatcagtgttgacccgaatgatcagaaaaagacagcttgttatgacattgatgttgaagtggatgacaccttgaagacccagatgaattcttttctgctgtccactgccagccaacaggagattgctactctagacaacaagacaatgactgatgtggtgggtaacccagaggaggagcgccgagctgagttctacttccagccctgggctcaggaggctgtgtgccgatacttctactccaaggtgcagcagagacgacaagaattagagcaagccctgggaatccggaatacatag

SEQ ID NO: 20 - DNA, 1068 bp, Homo sapien
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctga

(19) United States
(12) Patent Application Publication
(10) Pub. No.: US 2017/0165314 A1
(43) Pub. Date: Jun. 15, 2017

(54) AGENT CONTAINING IMIDAZOLE DIPEPTIDE

(71) Applicants: THE UNIVERSITY OF TOKYO, Tokyo (JP); KYUSHU UNIVERSITY, NATIONAL UNIVERSITY CORPORATION, Fukuoka-shi, Fukuoka (JP); NH FOODS LTD., Osaka-shi, Osaka (JP); NATIONAL CENTER OF NEUROLOGY AND PSYCHIATRY, Kodaira-shi, Tokyo (JP)

(72) Inventors: Tatsuhiro Hisatsune, Tokyo (JP); Mamoru Totsuka, Tokyo (JP); Hideo Satsu, Tokyo (JP); Jun Kaneko, Tokyo (JP); Yoshinori Katakura, Fukuoka (JP); Mikako Sato, Ibaraki (JP); Takashi Matsumoto, Ibaraki (JP); Fumiki Morimatsu, Hokkaido (JP); Etsuko Imabayashi, Saitama (JP); Hiroshi Matsuda, Saitama (JP)

(73) Assignees: THE UNIVERSITY OF TOKYO, Tokyo (JP); KYUSHU UNIVERSITY, NATIONAL UNIVERSITY CORPORATION, Fukuoka-shi, Fukuoka (JP); NH FOODS LTD., Osaka-shi, Osaka (JP); NATIONAL CENTER OF NEUROLOGY AND PSYCHIATRY, Kodaira-shi, Tokyo (JP)

(21) Appl. No.: 15/129,602

(22) PCT Filed: Mar. 4, 2015
(86) PCT No.: PCT/JP2015/056412

§ 371 Date: Sep. 27, 2016

(30) Foreign Application Priority Data
Mar. 28, 2014 (JP) 2014-069103
Jul. 11, 2014 (JP) 2014-142910

(51) Int. Cl. A61K 38/05; A23L 33/00; A23L 33/18; C12Q 1/37; C12Q 1/68

(52) U.S. Cl. CPC A61K 38/05; C12Q 1/37; C12Q 1/6883; C12Y 304/1302; A23L 33/18; A23L 33/30; C12Q 2600/158; G01N 2333/948; G01N 2800/2821; G01N 2800/52; G01N 2500/20; A23V 2002/00

(57) ABSTRACT

An anti-aging agent derived from a natural product is provided. The agent uses at least one selected from the group consisting of an imidazole dipeptide and a metabolite thereof as an active ingredient. The present invention also provides an agent for improving a neuropsychologic function, which contains at least one selected from the group consisting of an imidazole dipeptide and a metabolite thereof as an active ingredient. The present invention also provides an agent for changing expression of a transporter such as SLC23A2, which contains at least one selected from the group consisting of an imidazole dipeptide and a metabolite thereof, and an agent for controlling blood concentration of a cytokine such as IP-10, which contains at least one selected from the group consisting of an imidazole dipeptide and a metabolite thereof, as well as an expression analysis method for detecting improvement or degradation of a neuropsychologic function, and a kit for detecting improvement or degradation of a neuropsychologic function.

### TECHNICAL FIELD

The present invention relates to an agent using at least one selected from the group consisting of an imidazole dipeptide and a metabolite thereof as an active ingredient. The agent of the present invention can be used for improvement of cognitive function, improvement of mental function, anti-aging, and health maintenance. The present invention is useful in the fields of common food, health food, drug, beauty health, and medical science.

### BACKGROUND ART

Carnosine is a dipeptide consisting of β-alanine and histidine, and anserine is a dipeptide consisting of β-alanine and methylated histidine. It is known that both are contained in chicken etc. Carnosine and anserine have been investigated for their actions for promotion of skin metabolism (Patent document 1), autonomic nerve control (Patent document 2), and stress relieving (Patent document 3), as well as improvement of learning function and anti-anxiety (Patent document 4).

In recent years, things for living long with maintaining health, such as prevention of diseases accompanying aging and strengthening of immune activity, have come to attract growing interest. In many cases, aging is accompanied by decrease of cognitive function to a certain extent. Decrease of cognitive function is also caused by onset or advance of the Alzheimer disease.

### PRIOR ART REFERENCES

### Patent Documents

Patent document 1: Japanese Patent Unexamined Publication (Kokai) No. 2000-201649
Patent document 2: WO2002/076455
Patent document 3: Japanese Patent Unexamined Publication (Kokai) No. 2007-70316
Patent document 4: Japanese Patent. Unexamined Publication (Kokai) No. 2000-116987

### SUMMARY OF THE INVENTION

### Object to be Achieved by the Invention

It is desirable to delay advance of aging in the daily life with appropriate meals, exercises, psychotherapies, etc, and manage diseases accompanying aging at an early stage.

Foods are energy sources and sources of essential nutrients (nutritiousness), and in addition, they also give pleasure of eating (preference) and contribute to healthy life (functionality). Foods and products containing food-derived functional ingredients are expected to be useful for health or beauty, and they are generally and widely accepted as health foods etc. It is considered that use of already habitually eaten natural products is preferred also from the viewpoints of safety and no anxiety.

### Means for Achieving the Object

The inventors of the present invention have worked on researches and developments of foods and materials useful for health by utilizing natural products. In this process, they recently found that carnosine and anserine derived from chicken had a mental function-improving action etc., and accomplished the present invention. The present invention provides the followings.
[1] An agent for improving a neuropsychologic function, which contains at least one selected from the group consisting of an imidazole dipeptide and a metabolite thereof.
[2] The agent according to [1], wherein the neuropsychologic function is one relevant to the Alzheimer disease or aging.
[3] An anti-aging agent based on improvement of a neuropsychologic function, which contains at least one selected from the group consisting of an imidazole dipeptide and a metabolite thereof.
[4] An agent for changing expression of at least one kind of gene selected from the group consisting of:
transporter genes SLC23A2, SLC43A2, SLC29A3, SLC35C1, SLC25A33, SLC25A23, SLC6Al2, and SLC6A13;
chemokine genes CXCL12 and CCL17;
aging-related genes TSPO and P2RY1;
nervous system gene CAMK1;
mitochondrial genes ACO2, ATP7A, POLG, IDH3G, UCP2, BCKDHA, and TAP2; as well as
anti-aging genes SMARCD1 and SIRT6,
which contains at least one selected from the group consisting of an imidazole dipeptide and a metabolite thereof.
[5] The agent according to [1], which has an anti-inflammatory activity.
[6] An agent for controlling blood concentration of at least one kind of cytokine selected from the group consisting of IP-10 (CXCL10), IL-2, IL-5, IL-7, IL-8 (CXCL8), IL-13, G-CSF, and MCP-1 (CCL2), which contains at least one selected from the group consisting of an imidazole dipeptide and a metabolite thereof.
[7] The agent according to [1], which has a blood sugar level increase-suppressing action, or blood sugar level-decreasing action.
[8] The agent according to [1], which has a blood insulin concentration increase suppressing action, or blood insulin concentration-decreasing action.
[9] The agent according to any one of [1] to [8], which is for ingestion of 200 mg or more as a daily dose of at least one selected from the group consisting of an imidazole dipeptide and a metabolite thereof.
[10] A nutritional composition containing 200 mg or more as a daily dose of at least one kind of imidazole dipeptide derived from animal meat.
[11] The nutritional composition according to [10], wherein at least one kind of the imidazole dipeptide is derived from chicken.
[12] The agent or nutritional composition according to any one of [1] to [11], which further contains creatine and a nucleic acid.
[13] The agent or nutritional composition according to any one of [1] to [12], which is for an elderly person or a person with a slight mood disorder.
[14] An agent for a treatment of diabetes and/or Alzheimer disease, which contains at least one selected from the group consisting of an imidazole dipeptide and a metabolite thereof.
[15] A dieting method comprising making a subject for whom improvement of a neuropsychologic function is desirable ingest a nutritional composition containing at least one selected from the group consisting of an imidazole dipeptide and a metabolite thereof.
[16] A method for determining a neuropsychologic function of a subject, which is based on a carnosine decomposition enzyme (CNDP1) activity of the subject.
[17] A method for predicting a neuropsychologic function-improving effect brought by administering to a subject the agent or nutritional composition according to any one of [1] to [14], which is based on a CNDP1 activity of the subject.
[18] A method for detecting improvement or degradation of a neuropsychologic function, which is based on analysis of expression of at least one kind of gene selected from the group consisting of:
transporter genes SLC23A2, SLC43A2, SLC29A3, SLC35C1, SLC25A33, SLC25A23, SLC6Al2, and SLC6A13,
chemokine genes CXCL12 and CCL17;
aging-related genes TSPO and P2RY1;
nervous system gene CAMK1;
mitochondrial genes ACO2ATP7A, POLG, IDH3G, UCP2, BCKDHA, and TAP2; as well as
anti-aging genes SMARCD1 and SIRT6.
[19] A kit for detecting improvement or degradation of a neuropsychologic function, which comprises a nucleic acid comprising at least one kind of nucleotide sequence selected from an entire or partial sequence of any one of the nucleotide sequences of SEQ ID NOS: 1 to 20, and
an entire or partial sequence of a nucleotide sequence complementary to any one of the nucleotide sequences of SEQ ID NOS: 1 to 20.
[20] A pharmaceutical composition for a treatment of aging of cerebral function and/or dementia, which contains a compound represented by the formula I or II:

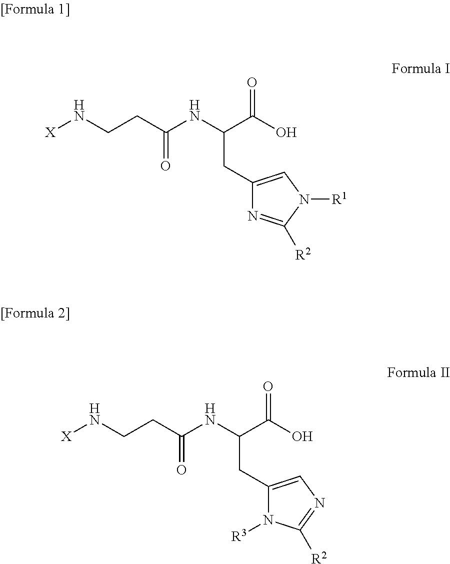

wherein, in the formulas I and II, R¹, R², and R³ are independently H or a C₁₋₆ alkyl, and X is H or —COR⁴, wherein R⁴ is H or a C₁₋₆ alkyl.
[21] A method for searching for an active ingredient for a treatment of aging of a cerebral function and/or dementia, which uses a compound represented by the formula I or II defined in claim 20 as a leading compound.

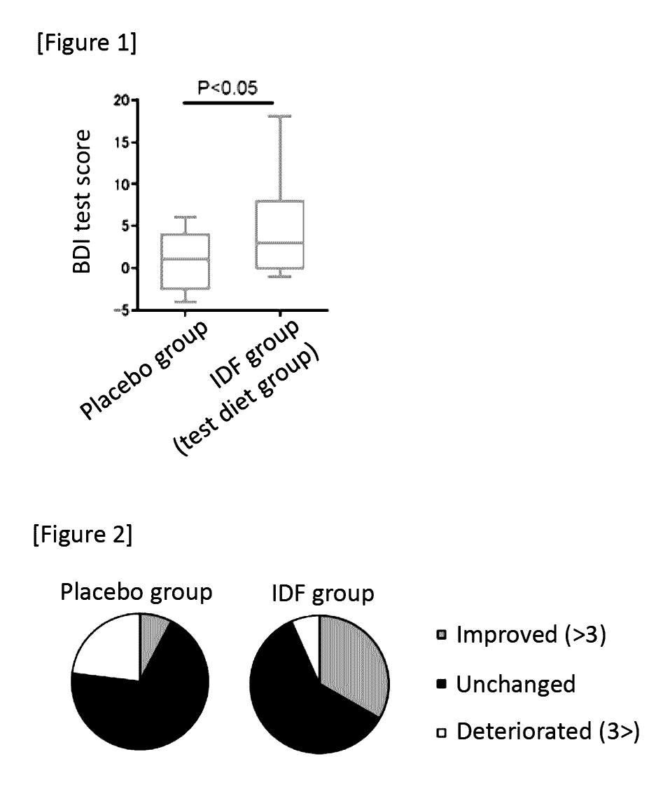

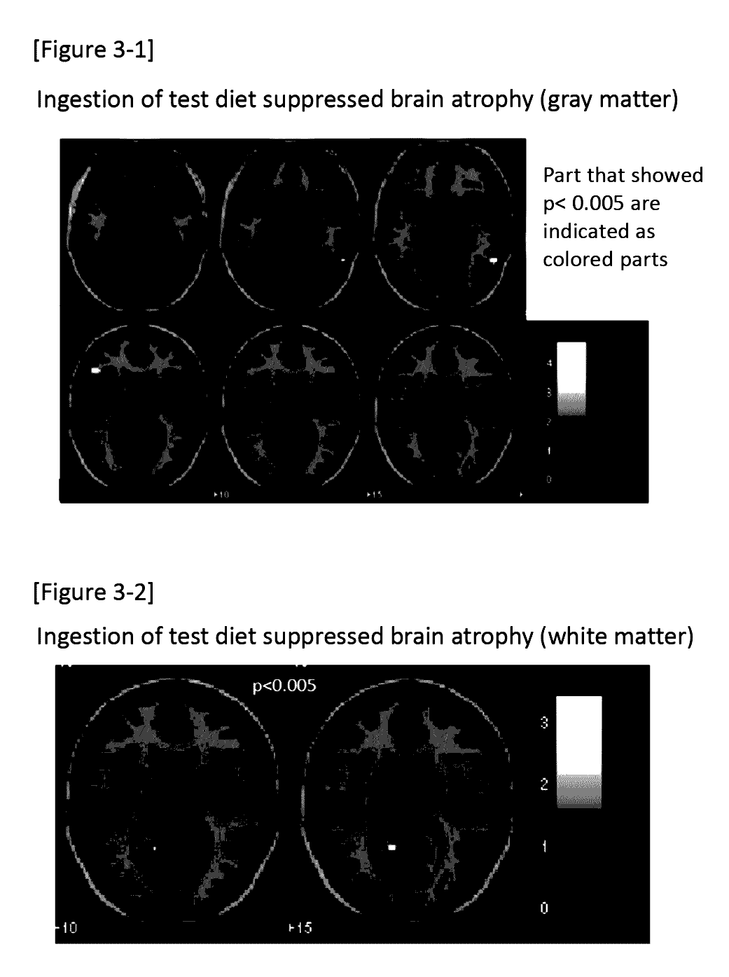

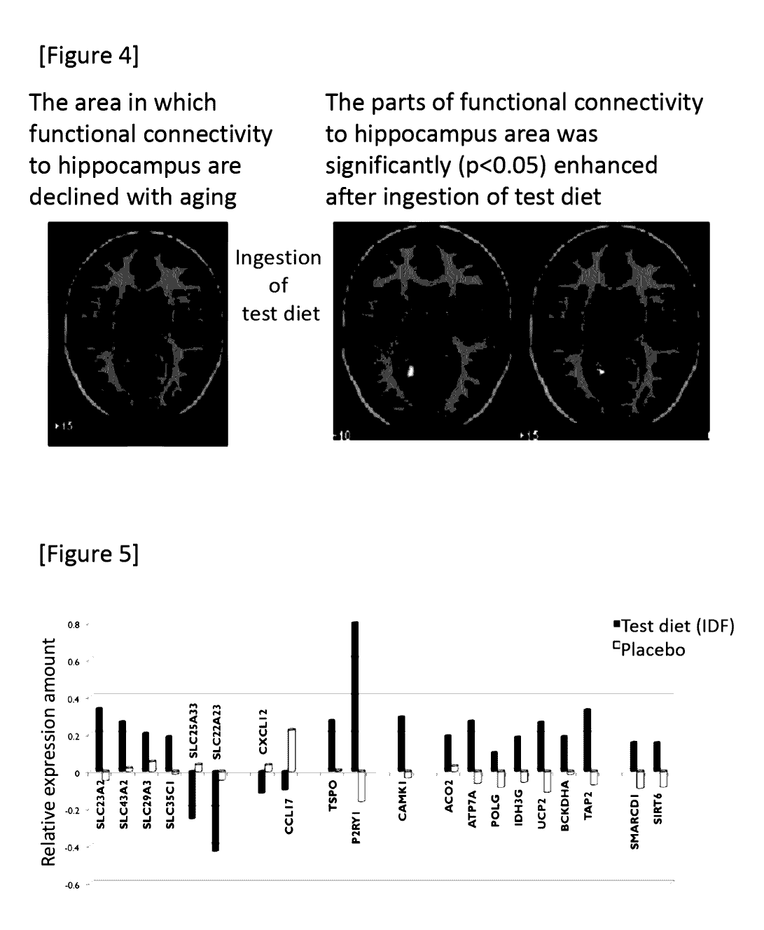

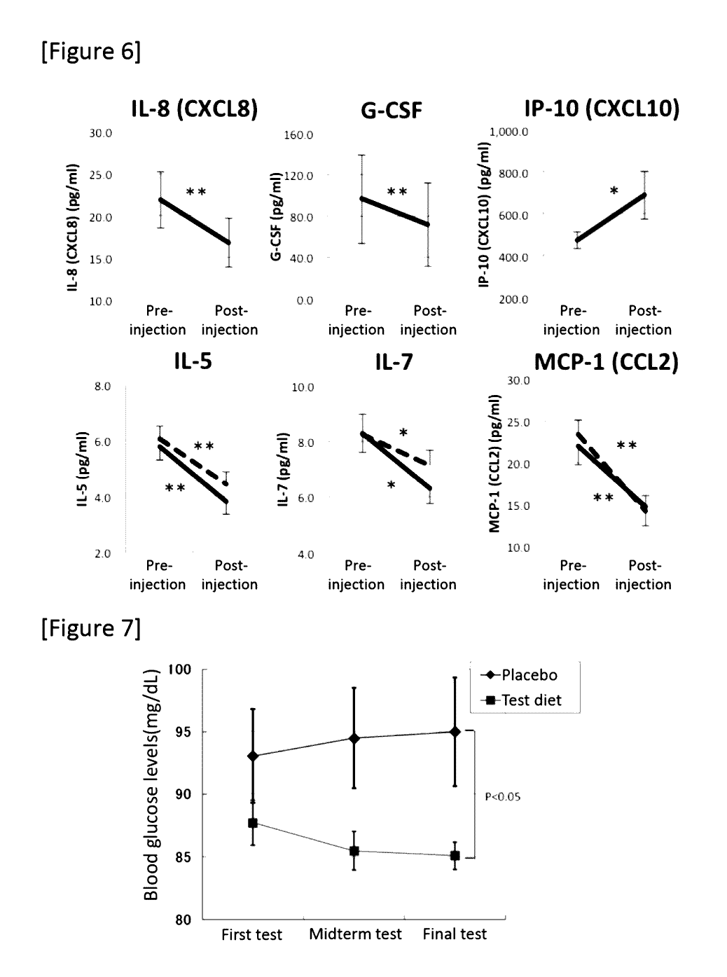

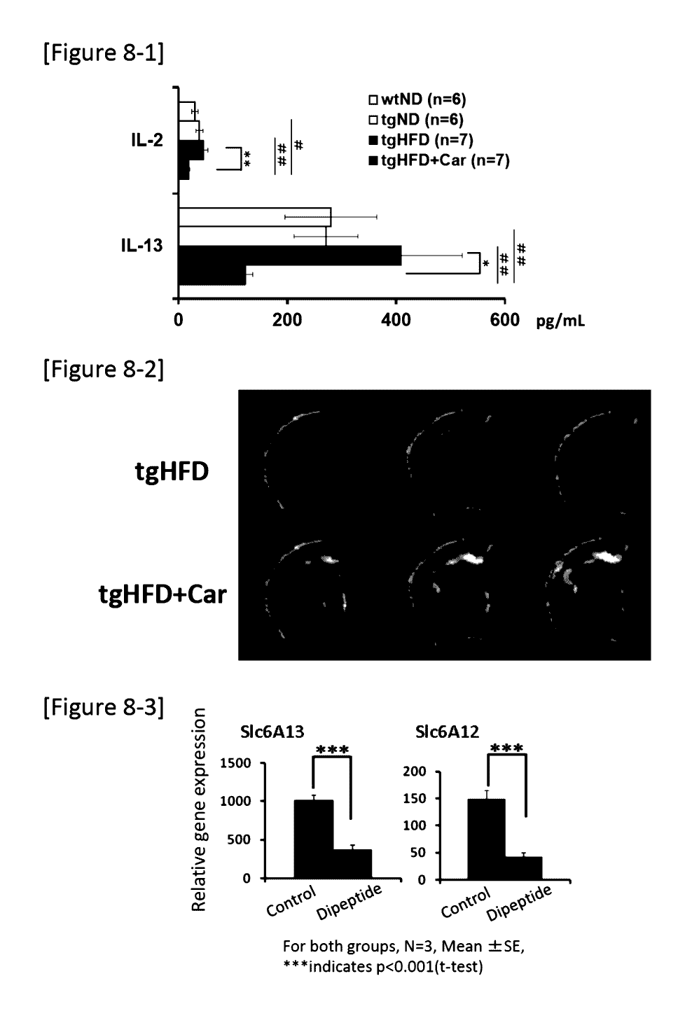

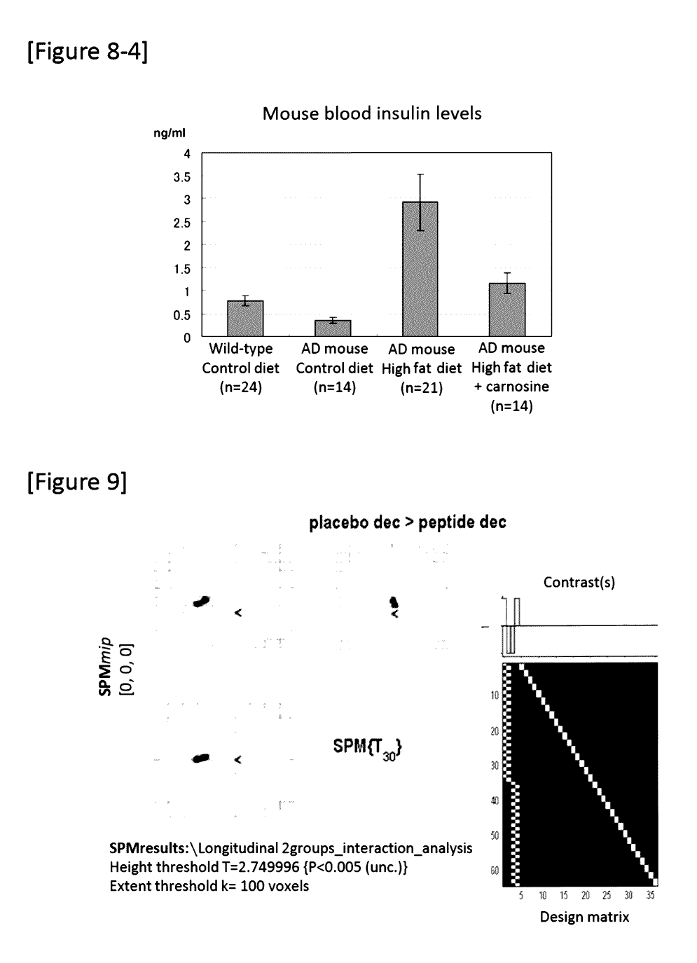

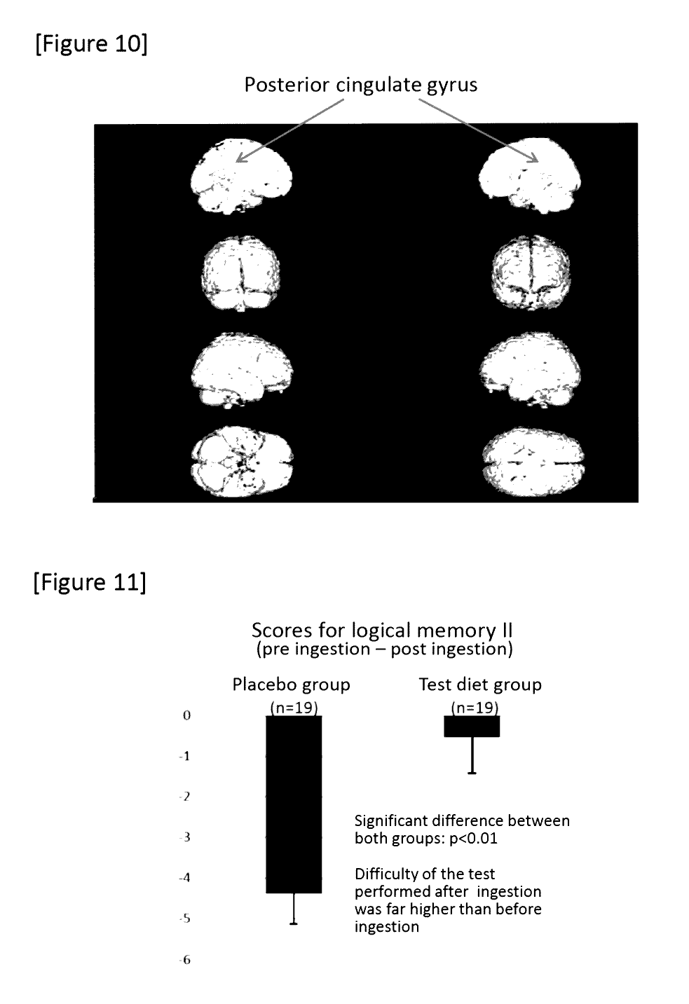

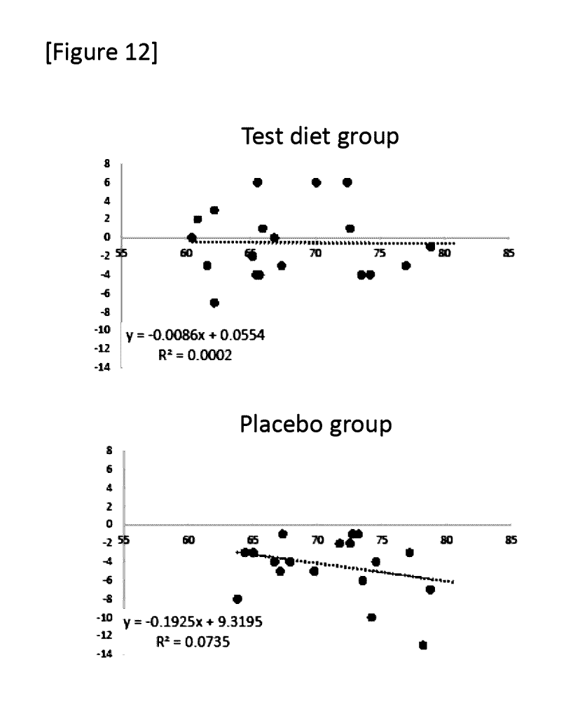

### BRIEF DESCRIPTION OF THE DRAWINGS

[FIG. 1] Results of a mental function test (BDI test)

[FIG. 2] Results of a mental function test (ADAS-cog test for evaluating aging of brain): ratios of improvement, no change, and degradation are indicated.

[FIG. 3-1] Parts of brain (gray matter) in which atrophy was improved by ingestion of an imidazole dipeptide: parts that showed p<0.005 are indicated as colored parts.

[FIG. 3-2] Parts of brain (white matter) in which atrophy was improved by ingestion of an imidazole dipeptide

[FIG. 4] Improvement of neural circuit function provided by ingestion of an imidazole dipeptide: although the neural circuit function of the hippocampus and posterior cingulate gyrus decreases in connection with aging (left), this decrease of function was improved in the test diet ingestion group after the ingestion (right).

[FIG. 5] Genes of which expression was changed by ingestion of an imidazole dipeptide: the gene expression levels of 6 genes belonging to SLC (transporter) were significantly changed by the ingestion of the test diet compared with that observed with ingestion of placebo food. In addition, it was also found that the expression levels of the other genes shown in the graph including chemokines genes also changed.

[FIG. 6] Blood cytokines and chemokines of which expressions were changed by ingestion of an imidazole dipeptide: the results are shown with average ±SE, the results of the test diet ingestion group are indicated with solid lines, the results of the placebo diet ingestion group are indicated with dashed lines, and the results of paired t-test are indicated with * (p<0.05), and ** (p<0.01).

[FIG. 7] Decrease of blood sugar level provided by ingestion of an imidazole dipeptide

[FIG. 8-1] Decrease of blood cytokines in dementia model mouse: in a carnosine-containing diet group, cytokines decreased, and it is suggested that inflammation was suppressed. The symbol wt stands for wild-type, tg for transgenic, * for p<0.05, ** for p<0.01 (Dunnett's test vs tgHFD), # for p<0.05, and ## for p<0.01 (Student t-test).

[FIG. 8-2] Suppression of intracerebral inflammatory reaction in dementia model mouse (mouse MRI images): suppression of inflammation provided by ingestion of an imidazole dipeptide was observed.

[FIG. 8-3] Suppression of expression of GABA transporter genes in glia cells provided by ingestion of highly functional dipeptide: Slc6A13 is GABA Transporter 2 (GAT-2) expressed in astrocytes, and Slc6A12 is Betaine/GABA Transporter 1 expressed in astrocytes (BGT-1).

[FIG. 8-4] Blood insulin levels in mice: between the values of the Alzheimer disease (AD) high fat diet group and the AD high fat diet+carnosine group, significant difference (student's t-test, p<0.05) were observed.

[FIG. 9] ASL analysis

[FIG. 10] Parts for which differences were observed between the both groups in the ASL analysis (posterior cingulate gyrus)

[FIG. 11] Scores for logical memory II (results of subanalysis for those 60 years old or older): there was significant difference between both groups (<0.01). Difficulty of the test performed after ingestion was far higher than that of the test performed before ingestion.

[FIG. 12] Scores for logical memory II (results of subanalysis for those 60 years old or older): function was not degraded for all the ages in the test diet group, whereas there was a tendency that severer hypofunction was observed for higher age in the placebo diet group.

### MODES FOR CARRYING OUT THE INVENTION

### Active Ingredient

The present invention relates to an agent that uses at least one selected from the group consisting of an imidazole dipeptide and a metabolite thereof as an active ingredient. The term imidazole dipeptide used in this specification refers to a dipeptide consisting of an amino acid having an imidazole ring, and another amino acid, which bind together, unless especially mentioned.

The imidazole dipeptide used in the present invention can be represented by following formula I or II.

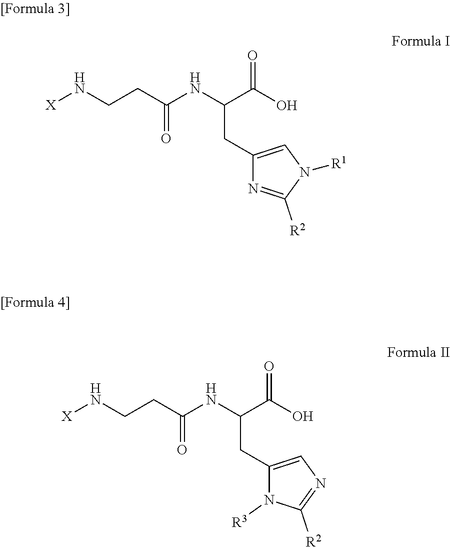

In the formulas I and II, R¹, R², and R³ are independently H or a C₁₋₆ alkyl, and X is H or —COR⁴, wherein R⁴ is H, a C₁₋₆ alkyl, benzyl which may be substituted, or H₂C═CH—.

In the formula I, it is preferred that one of R¹ and R² is a C₁₋₆ alkyl, and the other is H. In the formula II, it is preferred that one of R² and R³ is a C₁₋₆ alkyl, and the other is H. One of the preferred examples of C₁₋₆ alkyl is methyl.

Specific examples of —COR₄ as X include formyl, acetyl, propionyl, benzoyl, and acryloyl.

As for methods for producing the compounds represented by the formula I or II, and so forth, Japanese Patent Unexamined Publication (Kohyo) No. 2003-520221, Japanese Patent Unexamined Publication (Kokai) No. 2006-232686, Japanese Patent Unexamined Publication (Kohyo) Nos. 2006-504701, 2008-517911, 2009-512459, Japanese Patent Unexamined Publication (Kokai) Nos. 2010-31004, 2011-37891, 2011-37892, 2013-165728, 2014-12735, and so forth can be referred to.

Carnosine, anserine, balenine, and homocarnosine are included in the scope of the imidazole dipeptide. Carnosine is a dipeptide consisting of β-alanine and histidine. Depending on the conformation of the constituent histidine, carnosine exists as L-isomer or D-isomer. In the present invention and explanations thereof, when only the term “carnosine” is used, it refers to L-carnosine D-carnosine, or a mixture of them, unless especially indicated. It is known that L-carnosine exists in muscles and nervous tissues of mammals such as human at comparatively high concentrations.

The structure of L-carnosine (IUPAC nomenclature: (2S)-2-[(3-amino-1-oxopropyl)amino]-3-(3H-imidazol-4-yl)propanoic acid) is shown below.

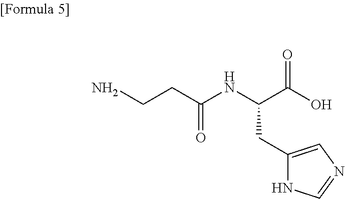 [SMILES: NCCC(=O)N[C@@H](Cc1cnc[nH]1)C(=O)O]

L-Anserine, which consists of β-alanine and methylated histidine, is abundantly observed in some animals. In the present invention and explanations thereof, when only the term “anserine” is used, it refers to L-anserine, D-anserine, or a mixture of them, unless especially indicated. The structure of L-anserine (IUPAC nomenclature: (2S)-2-[(3-amino-1-oxopropyl)amino]-3-(3-methyl-4-imidazolyl)propanoic acid) is shown below.

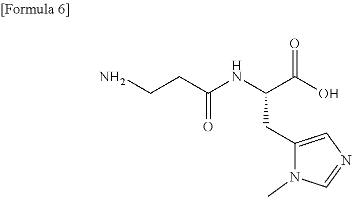 [SMILES: Cn1cncc1C[C@H](NC(=O)CCN)C(=O)O]

Both of carnosine and anserine are water-soluble (1 g/3.1 ml at 25° C. for carnosine).

The term metabolite of imidazole dipeptide used in this specification refers to one kind of metabolite selected from the group consisting of metabolites of carnosine, anserine, balenine, and homocarnosine, unless especially indicated. β-Alanine, histidine, methylated histidine, and γ-aminobutyric acid (GABA) are included in the scope of the metabolite of imidazole dipeptide.

The expression “at least one selected from the group consisting of an imidazole dipeptide and a metabolite thereof” used in the present invention is used to mean one or two or more kinds of imidazole dipeptides, metabolites thereof or both, unless especially indicated. For example, the expression “to contain at least one selected from the group consisting of an imidazole dipeptide and a metabolite thereof as an active ingredient” means, for example, to contain carnosine as an active ingredient, but not contain any other imidazole dipeptides, to contain carnosine and anserine as active ingredients, or the like. When quantity or concentration is referred to for “at least one selected from the group consisting of an imidazole dipeptide and a metabolite thereof”, and there are two or more kinds of imidazole dipeptides, metabolites thereof or both, the quantity or concentration refers to the total quantity or concentration of all the imidazole dipeptides, metabolites thereof or both. Although an explanation may be made in this specification for an embodiment using an imidazole peptide, or carnosine or anserine among the imidazole dipeptides and metabolites thereof, such an explanation also applies to embodiments using another imidazole dipeptide or a metabolite thereof.

In the present invention, the imidazole dipeptide and a metabolite thereof used as the active ingredient may be a synthesized one or one produced by fermentation, or one obtained from a natural product. They may be an isolated one, or purified one. More specifically, the imidazole dipeptide and a metabolite thereof may be one derived from any of various animals such as bovine, equine, porcine, fowl, whale, and fish (such as bonito, tuna, and eel). One of the preferred examples is one derived from chicken. The imidazole dipeptide or a metabolite thereof may be contained in the agent as an extract, concentrate, roughly purified product, or the like of a natural product.

### Use and Function

The agent of the present invention can be used for improvement of a neuropsychologic function. The improvement of a neuropsychologic function includes antidepressing (improvement of mental function) and improvement of cognitive function. The improvement of a neuropsychologic function also includes suppression of cerebral atrophy, suppression of cerebral hypofunction (enhancement of functional connectivity to the hippocampus), and improvement of damage of neurons caused by inflammation, which relate to a neuropsychologic function. The neuropsychologic function may be one relating to the Alzheimer disease or aging. The improvement of a neuropsychologic function also includes a treatment of aging of cerebral function and/or dementia.

The antidepressing (mental function-improving) effect provided by the agent of the present invention can be estimated by using the BDI questionnaire (http://www.chibatc.co.jp/catalogue/04/1/67.html). Since a high score of the BDI questionnaire represents a depressing tendency, degree of the improvement of a neuropsychologic function can be estimated by, for example, performing the inquiry before and after the ingestion of the agent of the present invention, and comparing the scores obtained before and after the ingestion. An effect of the agent of the present invention for improving a mental function can be expected especially for a subject having a slight mood disorder.

The effect of the agent of the present invention for improving cognitive function can be estimated by the ADAS-cog (Alzheimer's Disease Assessment Scale-cognitive subscale) method. The effect of the agent of the present invention for improving cognitive function includes those for improving the function degraded to such a degree that memory worsens with aging, and such a degree that cognitive ability degrades pathologically (dementia). The improving effect of the agent of the present invention can be expected especially for degradation of cognitive function relating to the Alzheimer disease or aging.

The agent of the present invention can be used for suppression of cerebral atrophy, suppression of cerebral hypofunction (enhancement of functional connectivity to the hippocampus), or improvement of damage of neurons caused by inflammation. The effects on these can be evaluated by the methods well known to those skilled in the art, such as diagnostic imaging. According to the examination of the inventors of the present invention, in a group of subjects including elderly persons, parts where advance of atrophy was suppressed were observed in both the cerebral gray matter and white matter. Such an effect was not observed in a group for which a placebo not containing anserine and carnosine was administered.

The agent of the present invention can be used for treating aging of cerebral function and onset of dementia, more specifically, for preventing, delaying, or inhibiting aging of cerebral function and onset of dementia, or preventing aggravation of these. Such an effect can be confirmed by evaluating blood flow change in the posterior cingulated gyrus of the subject, or evaluating logical memory (delayed word recall task) in the case of a subject 60 years old or older. According to the investigation of the inventors of the present invention, it was found that, in the case of subjects 60 years old or older, blood flow was maintained in the posterior cingulated gyrus in the imidazole dipeptide-containing test diet ingestion group in a degree significantly different from that observed for the placebo diet group. It was also found that, in the case of subjects 60 years old or older, the scores for the logical memory were maintained in the group of the subjects who ingested test diet for 3 months in a degree significantly different from that observed for the placebo diet group.

The agent of the present invention can be used for changing expression of a transporter. Transporters are membrane proteins existing on a cell membrane together with a channel or receptor. However, unlike channel, transporters recognize not only an endogenous substance, but also many exogenous substances including drugs and environmental chemical substances as a transportation substrate. Transporters are classified into two groups, ABC (ATP binding cassette) family, of which members carry out the transportation by using hydrolysis energy of ATP, and SLC (solute carrier) family, of which members carry out the transportation without using the energy of ATP, and 48 kinds of ABC transporter genes and 319 kinds of SLC transporter genes have been identified for human. Diseases caused by abnormalities of transporters increase with aging, and it is supposed that transporters relate to about 10% of genes relating to aging-associated diseases observed after 50 years old.

The agent of the present invention can be used especially for changing expression of at least one, preferably three or more, more preferably 5 or more, transporters selected from the group consisting of SLC23A2, SLC43A2, SLC29A3, SLC3SC1, SLC25A33, SLC25A23, SLC6A12, and SLC6A13, further preferably all of them. Changing expression includes increasing expression and decreasing expression.

The agent of the present invention can be used for changing expression of a chemokine. Chemokines are basic proteins that exhibit the actions thereof via a G-protein-coupled receptor, and constitute a group of cytokines. They induce migration of leucocytes, and so forth, and participate in formation of inflammation. Many chemokines have been discovered so far. According to structural difference, they are classified into CC chemokines, CXC chemokines, C chemokines, and CX3C chemokines, Not less than 50 kinds of chemokines have been identified so far.

The agent of the present invention can be used especially for changing expression of one selected from the group consisting of CXCL12 and CCL17, preferably both chemokines. Changing expression includes increasing expression and decreasing expression.

The agent of the present invention can be used for changing expression of an aging-related gene. Aging-related genes have been identified as genes involved in individual aging or cellular aging, and many aging-related genes have been identified so far.

The agent of the present invention can be used especially for changing expression of one selected from the group consisting of TSPO and P2RY1, preferably both aging-related genes. Changing expression includes increasing expression and decreasing expression.

The agent of the present invention can be used for changing expression of a nervous system gene. Nervous system genes are genes involved in neurogenesis nerve differentiation, and so forth, and many nervous system genes have been identified so far.

The agent of the present invention can be used especially for changing expression of the nervous system gene, CAMK1. Changing expression includes increasing expression and decreasing expression.

The agent of the present invention can be used for changing expression of a mitochondrial gene. Mitochondrial genes are genes involved in mitochondrial biosynthesis, fusion, TCA cycle, and respiration, and many mitochondrial genes have been identified so far.

The agent of the present invention can be used especially for changing expression of at least one, preferably 3 or more, more preferably 5 or more, mitochondrial genes selected from the group consisting of ACO2, ATP7A, POLG, IDH3G, UCP2, BCKDHA, and TAP2, further preferably all of them. Changing expression includes increasing expression and decreasing expression.

The agent of the present invention can be used for changing expression of an anti-aging gene. Anti-aging genes are genes that realize suppression of cellular aging and anti-aging, and many such genes have been identified.

The agent of the present invention can be used especially for changing expression of one selected from the group consisting of SMARCD1 and SIRT6, preferably both of them. Changing expression includes increasing expression and decreasing expression.

The agent of the present invention can also be used for controlling at least one kind of cytokine selected from the group consisting of IP-10 (CXCL10), IL-2, IL-5, IL-7, IL-8 (CXCL8), IL-13, G-CSF, and MCP-1 (CCL2). The controlling includes increasing the level and decreasing the level.

The agent of the present invention can also be used as an anti-inflammatory agent, or for suppressing increase of blood sugar level or decreasing the level.

The “improvement” or “treatment” referred to in the present invention for a disease or condition includes reducing risk of onset, delaying, preventing or treatment of onset, and arresting or delaying advance. Practices for the improvement or treatment include medical practices aiming at treatment of diseases performed by medical practitioners, and non-medical practices performed by those other than medical practitioners, such as dietitian (registered dietitian), hygienist, maternity nurse, nurse, medical technologist, cosmetic adviser, aesthetician, food manufacturer, and food vender. The treatment includes recommendation of administration or ingestion of specific foods, guidance for food ingestion method, health guidance, nutrition guidance (including guidance for nutrition required for medical treatment of sick persons, and guidance for nutrition for maintenance and promotion of health), food services, and guidance required for nutritional improvement relating to food dispensing. Objects of the treatment according to the present invention include humans (individuals), preferably humans who are desirably to be subjected to any one of the aforementioned treatments, or need to be subjected to any one of the aforementioned treatments.

The inventors of the present invention measured serum carnosine decomposition enzyme (henceforth referred to as CNDP1) activity of subjects for the purpose of verifying individual differences that affect the efficacy of imidazole dipeptide. As a result of the activity measurement, it was confirmed that there were significant individual differences among the subjects. CNDP1 exists in blood, and decomposes an imidazole dipeptide. Therefore, CNDP1 may affect the imidazole dipeptide concentration in blood after ingestion of the imidazole dipeptide, and thereby affect the efficacy of the imidazole dipeptide. Therefore, it is thought that information of the activity of CNDP1 in a subject is useful for judging beforehand whether the treatment by ingestion of an imidazole dipeptide is effective or not. Therefore, the present invention provides a method for determining a neuropsychologic function of a subject based on the carnosine decomposition enzyme (CNDP1) activity of the subject, and a method for predicting a neuropsychologic function-improving effect provided to a subject by making the subject ingest the agent or nutritional composition of the present invention based on the CNDP1 activity of the object. These methods may be such a method in which a standard value for judgment is defined beforehand, and the judgment or prediction is mechanically performed using the standard value.

### Agent

The term “agent” used in the present invention may refer to the active ingredient itself, or one containing the active ingredient and another ingredient, unless especially indicated. However, it does not include existing foods containing at least one selected from the group consisting of an imidazole dipeptide and a metabolite thereof, such as chicken itself.

The agent of the present invention may contain an ingredient other than the active ingredient, so long as it can exhibit the objective effect. The other ingredient may be any of various additives acceptable for foods, and various additives acceptable for drugs. Examples of such an ingredient include excipients, antioxidants (antioxidizing agents), perfumes, seasonings, sweeteners, coloring agents, thickening stabilizers, color developing agents, bleaching agents, antifungal agents, gum bases, bitter taste agents, enzymes, brighteners, acidulants, emulsifiers, enhancers, agents for manufacture, binders, tension agents (isotonic agents), buffering agents, dissolving aids, preservatives, stabilizers, coagulants, and so forth.

The other ingredient may be a functional ingredient other than the active ingredient. Examples of such a functional ingredient include amino acids (for example, branched chain amino acids, ornithine), unsaturated fatty acids (for example, EPA, DHA), vitamins, trace metals, glucosamine, and chondroitins.

When the agent of the present invention consists of the active ingredient and another ingredient other the active ingredient, content of the active ingredient can be appropriately determined by those skilled in the art from the viewpoints of ease of manufacture, ease of use, etc., and it may be, for example, 0.1 to 99.9%, 1 to 95%, 10 to 90%, or 51 to 90%. Content of carnosine may be 21% or higher, and content of Anserine may be 31% or higher.

As described above, form of the agent of the present invention may be any of various forms other than those of existing foods. For example, it may be a pharmaceutical composition such as an Oral drug, or nutritional composition. The agent of the present invention can be used by adding it to a pharmaceutical composition such as an oral drug, or nutritional composition. The “nutritional composition” referred to in the present invention includes not only a solid one, but also one in the form of liquid, such as drink, unless especially indicated. The “nutritional composition” referred to in the present invention also includes health food, supplement, and food with health claims (including food with nutrient function claims and food for specified health uses), as well as dietetic food (one that achieves the purpose of treatment, which is cooked according to a menu prepared by a dietitian or the like according to a dietary slip prepared by a medical practitioner), food for alimentary therapy, homogenized food, low salt diet, care food, decreased calorie diet, diet food, and materials for these, unless especially indicated.

Examples of the form of the agent of the present invention, the pharmaceutical composition, and the nutritional composition include powder, subtilized granule, granule, tablet, capsule, liquid preparation (including elixir, lemonade, syrup, emulsion, suspension, solution, and drinkable preparation), gelatinous formulation, dietetic food, drink, confectionery, meat product, marine product-processed product, vegetable-processed product, daily dish, seasoning composition, and food additive.

Ingestion amount of the active ingredient of the present invention can be appropriately determined by those skilled in the art according to age, weight, sex, disease or condition to which the agent is applied, and so forth of the subject who ingests it. Ingestion amount of the active ingredient may be, for example, 200 mg/day or more, preferably 400 mg/day or more, more preferably 500 mg/day or more, further preferably 750 mg/day or more. It may also be 1,000 mg/day or more, 2,000 mg/day or more, 5,000 mg/day or more, or 7,500 mg/day or more. In any case, it can be 10,000 mg/day or less. Irrespective of how the minimum amount is defined, it may be 50,000 mg/day or less, preferably 30,000 mg/day or less, more preferably 20,000 mg/day or less, more preferably 10,000 mg/day or less. The active ingredient of the aforementioned daily ingestion amount may be ingested at one time, or at a plurality of times as divided portions.

Although the amount of the active ingredient contained in the agent, pharmaceutical composition, or nutritional composition of the present invention can be appropriately determined by those skilled in the art, it may be, for example, 1,000 mg/100 g or more, preferably 1,500 mg/100 g or more, more preferably 2,000 mg/100 g or more, still more preferably 2,500 mg/100 g or more, further preferably 3,000 mg/100 g or more, still further preferably 3,500 mg/100 g or more. Irrespective of the minimum amount, it may be 50,000 mg/100 g or less, preferably 40,000 mg/100 g or less, more preferably 30,000 mg/100 g or less, further preferably 20,000 mg/100 g or less.

The agent, pharmaceutical composition, or nutritional composition of the present invention may also contain an ingredient other than the active ingredient. The ingredient other than the active ingredient is, for example, creatine or a nucleic acid. Content of creatine can be, for example, 10 mg or more, preferably 20 mg more, more preferably 30 mg or more, still more preferably 60 mg or more, further preferably 100 mg or more, still further preferably 200 mg or more, in the daily dose. Irrespective of the minimum amount, it can be 2,000 mg or less, preferably 1,000 mg or less, more preferably 750 mg or less, further preferably 500 mg or less. Content of a nucleic acid can be, for example, 0.15 mg or more, preferably 0.30 mg or more, more preferably 0.50 mg or more, still more preferably 1.0 mg or more, further preferably 2.0 mg or more, still further preferably 3.0 mg or more, in the daily dose. Irrespective of the minimum amount, it can be 50 mg or less, preferably 40 mg or less, more preferably 20 mg or less, further preferably 10 mg or less.

When the agent, pharmaceutical composition, or nutritional composition of the present invention is used as a dietetic food (one that achieves the purpose of treatment, which is cooked according to a menu prepared by a dietitian or the like according to a dietary slip prepared by a medical practitioner), food for alimentary therapy, homogenized food, low salt diet, care food, decreased calorie diet, diet food, or sports food (including food aiming at enhancement of ability in aerobic exercise, food aiming at enhancement of endurance in aerobic exercise, food for accumulating nutrition in the body by the day of game, food for supplementing nutrition during game, and food aiming at recovery from exhaustion after finishing game), content of the active ingredient can be determined in consideration of ingestion amount as one meal.

The agent, pharmaceutical composition, or nutritional composition of the present invention can be ingested by a subject repeatedly or over a long period of time. Especially when enhancement of exercise capacity is intended, it will be preferable to make the subject ingest it before exercise, or ingest daily.

For the agent, pharmaceutical composition, or nutritional composition of the present invention, it can be indicated that it can be used for improvement of a neuropsychologic function, suppression of cerebral atrophy, suppression of cerebral hypofunction, and improvement of damage of neurons caused by inflammation, and it can also be indicated that ingestion thereof is recommended to, for example, elderly people 65 years old or older, or persons having a slight mood disorder. The indication may be a direct indication, or an indirect indication. Examples of the direct indication include a statement on articles such as the product itself, package, container, label, and tag, and examples of the indirect indication includes advertising and public relations on such places or by such means as website, shop front, exhibition, signboard, bulletin board, newspaper, magazine, television, radio, postal matter, and E-mail.

### Production Method

The agent, pharmaceutical composition, or nutritional composition of the present invention can be produced by using various known techniques. The step of adjusting concentration of the active ingredient to a predetermined concentration may be performed in various stages of the manufacturing process. Those skilled in the art can appropriately design the steps for producing the agent of the present invention in consideration of solubility, stability, volatility, etc. of the active ingredient. According to the investigations of the inventors of the present invention, it was confirmed that anserine and carnosine are sufficiently stable at an ordinary temperature, and they are also sufficiently stable under the cooking conditions of 180° C. or lower. It was also confirmed that they can be stably stored over at least 2 years and 9 months in a state of solution.

When the active ingredient of the present invention is constituted as a chicken extract, an example of the method for producing such a chicken extract specifically comprises mincing chicken, adding warm water to the chicken, adjusting pH of the mixture if needed, and allowing extraction over several minutes to several days with warming as required. As for the extraction conditions, the treatment is performed, for example, at 50 to 100° C. for 1 to 10 hours. The obtained extract can be purified or fractionated by diatomaceous earth filtration, ultrafiltration, or the like, as required. The product can be subjected to a demineralization treatment and protease treatment as required. Although the part from which the chicken as the raw material is obtained is not particularly limited, it preferably contains breast meat, since it abundantly contains carnosine and/or anserine. The obtained extract can be dried into a dry product by hot air drying, spray drying, lyophilization, or the like. It can also be granulated into granules.

### Expression Analysis

The present invention also provides a method for detecting improvement or degradation of a neuropsychologic function, which is based on analysis of expression of at least one kind of gene selected from the group consisting of transporter genes SLC23A2, SLC43A2, SLC29A3, SLC35C1, SLC25A33, SLC25A23, SLC6A12, and SLC6A13; chemokine genes CXCL12 and CCL17; aging-related genes TSPO and P2RY1; nervous system gene CAMK1; mitochondrial genes ACO2, ATP7A, POLG, IDH3G, UCP2, BCKDHA, and TAP21 as well as anti-aging genes SMARCD1 and SIRT6, and a kit for detecting improvement or degradation of a neuropsychologic function, which comprises a nucleic acid comprising at least one kind of nucleotide sequence selected from an entire or partial sequence of any one of the nucleotide sequences of SEQ ID NOS: 1 to 20, and an entire or partial sequence of a nucleotide sequence complementary to any one of the nucleotide sequences of SEQ ID NOS: 1 to 20.

The expression analysis is preferably performed by analyzing expression of at least one kind of transporter gene selected from the group consisting of SLC23A2, SLC43A2, SLC29A3, SLC35C1, SLC25A33, SLC25A23, SLC6.Al2, and SLC6,A13at least one kind of chemokine gene selected from the group consisting of CXCL12 and CCL17, at least one kind of aging-related gene selected from the group consisting of TSPO and P2RY1, a nervous system gene selected from CAMK1, at least one kind of mitochondrial gene selected from the group consisting of ACO2, ATP7A, POLG, IDH3G, UCP2, BCKDHA, and TAP2 , and at least one kind of anti-aging gene selected from the group consisting of SMARCD1 and SIRT6. More preferably, the expression analysis is performed by analyzing expression of all of these genes.

In the development of ingredients of new drugs, functional food compositions, etc., actions of candidate drugs or ingredients are monitored at the cellular level to evaluate efficacy and safety thereof, and a technique of quantifying genes expressed in cells before and after administration of a drug or ingredient for the entire genome region, and quantitatively grasping action of the drug or ingredient as change of expression amounts of the genes attracts attentions. By analyzing expressions of a combination of genes defined in the present invention using such a method as mentioned above, effects of a candidate drug on a neuropsychologic function can be analyzed.

In the aspects of the present invention concerning expression analysis, the nucleic acid comprising at least one kind of nucleotide sequence selected from an entire or partial sequence of any one of the nucleotide sequences of SEQ ID NOS: 1 to 20, and an entire or partial sequence of a nucleotide sequence complementary to any one of the nucleotide sequences of SEQ ID NOS: 1 to 20 may be a probe that can specifically hybridize with a transcript in a sample as an object of detection, or a pair of primers that can function as primers for amplifying all or a part of such a transcript as mentioned above. The nucleic acid may be DNA or RNA.

In the aspects of the present invention concerning expression analysis, length of the nucleic acid comprising at least one kind of nucleotide sequence selected from an entire or partial sequence of any one of the nucleotide sequences of SEQ ID NOS: 1 to 20, and an entire or partial sequence of a nucleotide sequence complementary to any one of the nucleotide sequences of SEQ ID NOS: 1 to 20 used as the probe is, for example, 15 nucleotides length or longer, preferably 20 nucleotides length or longer, more preferably 25 nucleotides length or longer. In order to enable detection and quantification of a target nucleic acid, the probe nucleic acid may be labeled with, for example, a radioisotope, enzyme, fluorescent substance, or luminescent substance. The nucleic acid used as the probe may be immobilized on a solid phase.

Length of the nucleic acids used as primers is, for example, 15 to about 100 nucleotides length, preferably 15 to 50 nucleotides length, and they preferably consist of a pair of nucleotide sequences designed so that they can amplify a DNA fragment of 100 bp to several kbp.

A nucleic acid to be used can be prepared by chemical synthesis using a commercial automatic DNA/RNA synthesizer, or the like. A chip (array) on which a nucleic acid is immobilized can also be prepared by synthesizing the nucleic acid directly on a solid phase of silicon, glass, or the like. Preferred examples of embodiment in which the nucleic acid probe is immobilized on a substrate include a DNA microarray.

For quantitatively analyzing expression of predetermined genes using a small amount of sample, competitive RT-PCR real-time can be used. The sample as the object of the analysis may be blood collected from human.

### Use as Leading Compound

The present invention provides a method for searching for an active ingredient or the like for improvement of a neuropsychologic function, especially a treatment of aging of a cerebral function, and/or dementia, etc. (screening method) by using the aforementioned imidazole dipeptide or a metabolite thereof as a leading compound. The term leading compound generally refers to a compound of which profiles of pharmacological activities have been clarified, and for which improvement of activity or reduction of toxicity is expected as a result of chemical modification thereof. The imidazole dipeptide and a metabolite thereof have pharmacological activities for improvement of a neuropsychologic function, especially a treatment of aging of a cerebral function and/or dementia as described above, and it is expected that improvement of the activity or reduction of toxicity is provided by chemical modification thereof.

The chemical modification means, for example, optimization of a leading compound through chemical modification of the leading compound. The chemical modification can be, for example, substitution or deletion of a part of amino acids, or addition or insertion of at least one amino acid. There are also contemplated addition, substitution or removal of a functional group on an amino acid, and substitution of D-amino acid or artificial amino acid for an amino acid.

By performing optimization using the imidazole dipeptide or a metabolite thereof as a leading compound according to the present invention, an active ingredient showing further superior physicochemical properties, pharmacokinetics, toxicity, etc. can be searched for.

Hereafter, the present invention will be explained with reference to examples. However, the scope of the present invention is not limited by the following examples.

### EXAMPLES

### Evaluation 1 with Healthy Volunteers

Test diets containing chicken-derived imidazole dipeptides (1000 mg as daily dose of carnosine and anserine) were given to test subjects (28 healthy male or female volunteers 40 years old or older, divided into two groups of test diet group and placebo diet group) over 3 months, and change of the cerebral function etc. were evaluated before, during, and after the test period. Compositions of the test diet and placebo diet are shown in the following tables (as daily amounts).

TABLE 1

| Test diet | | (mg) |
|---|---|---|
| Chicken | Imidazole dipeptide (anserine + carnosine) | 1000 |
| extract | (as histidine | 210) |
| | Lysine | 57 |
| | Carbohydrate | 500 |
| | Moisture | 125 |
| | Ash | 35 |

| Excipient (dextrin, cornstarch, lactose) | 3590 |
|---|---|
| Total | 6000 |
| Placebo diet | (mg) |
| Imidazole dipeptide (anserine + carnosine) | 0 |
| Histidine | 210 |
| Lysine | 57 |
| Carbohydrate | 500 |
| Moisture | 125 |
| Ash | 35 |
| Excipient (dextrin, cornstarch, lactose) | 5733 |
| Total | 6000 |

### 1. Antidepressing Effect (Evaluation Using BDI Questionnaire)

Before and after the ingestion period, depressing tendency was evaluated with the BDI questionnaire (http://www.chibatc.co.jp/catalogue/04/1/67.html). A higher score of the BDI questionnaire indicates higher depressing tendency.

The results are shown in FIG. 1. Degrees of the improvement were compared on the basis of change of BDI score, i.e., score obtained before ingestion (test 1)−score obtained after ingestion (test 2). As a result, improvement tendency was seen for the test diet group, whereas improvement was hardly seen for the placebo group. Further, the changes before and after ingestion were ranked (a larger number of ranking indicates higher improvement). As a result, there was observed a tendency that the test diet group showed larger change. Even healthy subjects may have a slight depressing tendency, and it is considered that the imidazole dipeptide improved such a tendency by making functions of the GABA nervous system complete.

### 2. Cognitive Function-Improving Effect (Evaluation Using ADAS-Cog)

Cognitive functions were evaluated before and after the ingestion period by using ADAS-cog (Alzheimer's Disease Assessment Scale-cognitive subscale).

The results are shown in FIG. 2. Ratios of the subjects whose scores were improved or degraded by 3 points or more are shown. The ratio of subjects whose scores were improved of the test diet group was larger than that of the placebo group.

### 3. Effect on Cerebral Atrophy etc.

Brain structure analysis based on three-dimensional T1-weighted image and functional connectivity analysis based on resting-state functional MRI were performed. As a result of longitudinal analysis for base line and structural change after three months performed for 15 subjects of the test diet group and 13 subjects of the placebo diet group, it was found that advance of atrophy was more suppressed in the test diet group compared with the placebo diet group in the right inferior frontal gyrus and left inferior temporal gyrus for the gray matter (FIG. 3-1), and in the right posterior cingulate gyrus for the white matter (FIG. 3-2).

In the functional connectivity analysis based on resting-state functional MRI, it was found for the baseline that the functional connectivity to the hippocampus was reduced in the posterior cingulate gyrus with aging (FIG. 4). It is known that the posterior cingulate gyrus participates in reproduction of memory, and the function thereof reduces first in the Alzheimer disease. In the test diet group, the functional connectivity to the hippocampus of this part was enhanced three months afterward compared with the placebo diet group. This part coincided with the part of the white matter where the atrophy-suppressing effect was seen in the test diet group (FIG. 3-2).

### 4. Gene Expression Analysis

Blood samples of 13 subjects of the test diet group (samples could not be prepared for 2 subjects) and all the 13 subjects of the placebo group (those at the time of the first test and midterm test) were collected by using PAXgene RNA blood collection tubes (Nippon Becton Dickinson Company, Ltd., Tokyo), high-quality RNA was prepared by using PAXgene Blood RNA kit (Qiagen), and change of gene expression was analyzed by using a microarray.

### Methods

Whole Human Genome Oligo DNA Microarray (4×44K) V2 (Agilent, Calif., USA) was used as microarray.

### (1) Labeling

First, total RNA was extracted from each blood sample of test subject by using PAXgene Blood RNA Kit (Qiagen), and 200 ng of each total RNA was labeled by using Agilent Low-Input QuickAmp Labeling Kit, One-color. First, 200 ng of the total RNA in a volume of 2.5 μL was added to 2 μL of One-Color Spike Mix stock solution prepared beforehand. Then, 0.8 μL of T7 Promoter Primer was added, the mixture was incubated at 65° C. for 10 minutes on a heat block, and then quenched on ice for 5 minutes. Then, 4.7 μL, of cDNA Master Mix prepared beforehand was further added, and the mixture was incubated on a heat block at 40° C. for 2 hours, and moved onto a heat block at 70° C. for further incubation for 15 minutes. Then, the mixture was quenched for 5 minutes on ice, and 6 μL of Transcription Master Mix prepared beforehand was added to the mixture. The mixture was incubated for 2 hours on a heat block at 40° C. with light shielding, then the total volume of the mixture was made to be 100 μL by addition of 84 μL, of nuclease-free water, 350 μL of Buffer RLT was further added to the mixture, and 250 μL of ethanol was further added to the mixture. Then, the total volume of the mixture was applied to an RNeasy column, centrifuged at 4° C. and 13000 rpm for 30 seconds, washed twice with 500 μL of a buffer RPE, and finally eluted with 30 μL of RNase-free water.

### (2) Hybridization

Then, hybridization was performed according to the protocol recommended by Agilent. First, RNA eluted above was fragmented by mixing it with Fragmentation mix, incubated for 30 minutes on a heat block at 60° C., and immediately cooled on ice for 1 minute. Then, cRNA from Fragmentation Mix was mixed with 2×GE×Hybridization Buffer HI-RPM to prepare a hybridization mix. The hybridization mix was applied to the microarray slide, the slide was disposed in a hybridization chamber, and then disposed in a hybridization oven, and hybridization was allowed at 65° C. and 10 rpm for 17 hours.

### (3) Washing and Scanning of Microarray Slide

The microarray slide was washed by using Gene Expression Wash Buffer prepared beforehand. First, before the end of the hybridization, Gene Expression Wash Buffer 1 was filled in two glass containers for washing, and Gene Expression Wash Buffer 2 at 37° C. was filled in another one glass container (total three glass containers for washing). After the end of the hybridization, the hybridization chamber was disassembled in the first glass container for washing, and the microarray slide was taken out, and washed in the second glass container for washing. The microarray slide was further washed in the third glass container for washing, then slowly pulled up from the water surface so that it was dried, and finally mounted on a scanner for exclusive use, and the microarray slide was scanned.

### (4) Data Analysis

Data were converted into numerals with Feature Extraction software of Agilent. Normalization was performed according to the quantile method by using the statistical analysis software R. Z-Scores and ratios were calculated from the normalized signal values, and signal values showing variation of ±2 or larger were extracted. The obtained data were analyzed by using the annotation database DAVID (http://david.abcc.ncifcrf.gov/).

First, GenBank Accession Numbers of genes for which change was confirmed were inputted into the database, and then clustering was performed for every function for which genetic change occurred by performing functional annotation clustering. Pathway analysis of KEGG (Kyoto Encyclopedia of Genes and Genomes) was similarly conducted by using DAVID.

### Results

Genes that showed significant change due to the ingestion of the test diet compared with the placebo diet group are shown in FIG. 5, which were determined on the basis of significant difference level p<0.05. From the results of this gene expression analysis, it was found that expressions of various kinds of transporter molecules existing on blood cells significantly changed. It was found that, in particular, expression amount of the vitamin C transporter, which exists on the membrane surface of lymphocyte, was significantly increased by the ingestion of the imidazole dipeptide (SLC23A2 in FIG. 5). Expressions of a plurality of genes relating to energy metabolism of mitochondria (ACO2 (aconidase) and IDH3G (isocitrate dehydrogenase), which are enzymes of TCA cycle) were increased. The imidazole dipeptide may exhibit the health promotion action by using such a mechanism.

As for the chemokines, decreases of expressions of the CXC chemokines and CC chemokines were observed. It is suggested that the test diet tends to suppress inflammation.

Enhancement of expressions of aging-related genes was also observed. Suppression of aging by the test diet is suggested.

Enhancement of expressions of anti-aging genes was also observed. Anti-aging action of the test diet is suggested.

Muscle fatigue recovery effect of carnosine is known, and it has been considered that the recovery of fatigue is an effect of neutralization of muscular pH. However, since enhancement of genes of mitochondrial system was observed with ingestion of the test diet in the above tests, a novel function thereof exerted for muscles of enhancing mitochondrial functions via the glycolysis system is also suggested. It was also clarified by the above tests that expression of SIRT6 known as a long-life gene was enhanced. It is known that if this gene is overexpressed in a mouse individual, life of the mouse is prolonged. Therefore, it may also be expected that prolongation of life can be provided by use of an imidazole dipeptide. Further, concerning change of expressions of various SLC genes provided by ingestion of the imidazole peptide, a so-called food combination effect may also be expected, that is, it may be expected that if it is eaten together with carnosine, responses of them to various physiologically active substances and food ingredients change.

### 5. Change of Serum Cytokine Concentration

Biochemical test, blood cell count test, blood sugar test, and coagulation test were conducted for blood samples obtained from the test subjects before, during (six weeks after the start of ingestion), and immediately after the food ingestion period of three months. When the test diet was given, a blood sugar level-decreasing tendency was observed. The other indexes were not changed before and after the ingestion, and therefore safety of ingestion of the test diet and placebo diet was reconfirmed.

Quantitative analysis of 27 kinds of cytokines and chemokines was carried out for the same blood samples. Quantitative analysis of cytokine concentrations in sera of peripheral bloods of test subjects was conducted by the bead-based multiplex analysis using xMAP technology (Luminex). In this method, cytokines are simultaneously quantitatively analyzed by using specific antibodies bound to beads labeled with different fluorescent substances according to the principle of flow cytometry. The outline of the analysis method using the Bio-Plex Pro™ Human Cytokine Grp I Panel 27-pLex kit (Bio-Rad) is explained below. Antibody beads of different types were put into wells of a 96-well assay plate, and washed twice with the Bio-Plex Wash buffer. Then, serum and a standard solution were added to each well, and the plate was incubated on a shaker at room temperature for 1 hour with shielding. light. The beads were washed 3 times with the Wash buffer, then detection antibody solutions were added, and the plate was incubated on a shaker at mom temperature for 30 minutes with shielding light. The beads were washed 3 times with the Wash buffer, then a PE-labeled streptavidin solution was added, and the plate was incubated on a shaker at room temperature for 10 minutes with shielding light. The beads were washed 3 times with the Wash buffer, then the Assay buffer was added, and the plate was shaken for 10 seconds with shielding light. PE fluorescence intensity of each type of beads was measured by using Bio-Plex 200 System (Bio-Rad), and concentration of each cytokine in the serum was obtained by using a standard curve created with samples of known amounts. Statistical analysis was performed by paired t-test for the data obtained before and after the ingestion for every test subject, and cytokines for which the test diet group showed difference are shown in FIG. 6.

It was found that blood level of many kinds of cytokine and chemokine molecules including IL-8 (CXCL8) as well as IL-5, IL-7, granulocyte colony-stimulating factor (G-CSF), MCP-1 (CCL2) etc. were significantly reduced by the ingestion of the test diet. It was also found that, on the other hand, the blood level of IP-10 (CXCL10) was significantly increased by the ingestion of the test diet. The placebo diet used in this test contained histidine so that it contained the same amount of essential amino acids as that of the test diet, and it is known that histidine has an anti-inflammatory activity. Therefore, among the aforementioned molecules, blood concentrations of IL-5, IL-7, and MCP-1 (CCL2) were also significantly reduced by the ingestion of the placebo diet.

The blood sugar levels were measured. The results are shown in FIG. 7. The placebo group showed an increasing tendency, and the test diet group showed a decreasing tendency. Thus, the test diet group showed a blood sugar level-improving tendency compared with the placebo group. Since this pilot test was conducted with healthy middle and old age people as the subjects, concentrations of HbA1c (saccharified hemoglobin), which is used as a marker molecule of diabetes, were within a normal range in most of the test subjects, and the concentrations were not changed by the ingestion of the test diet.

### Evaluation with Pathological Mice

High fat diet (HFD) was given to transgenic mice (Alzheimer disease model mice) to induce cerebral hypofunction. Carnosine (L-histidine-β-alanine) was administered to the mice, and influence of carnosine was evaluated.

The results are shown in FIGS. 8-1, 8-2, 8-3, and 8-4. In the carnosine-containing diet group, cytokines decreased, and suppression of inflammation was suggested (FIG. 8-1). Suppression of cerebral inflammation in the carnosine administration group was also shown by the results of MRI test (FIG. 8-2, red portions). The results of the microarray analysis indicated increase of expressions of GABA transporters such as 7S1c6a12 and slc6a13 observed in the Alzheimer model was suppressed in the carnosine administration group (FIG. 8-3). The amount of GABA that can act as a transmitter is decreased by increase of expressions of the transporters in the Alzheimer disease model mice, and there was suggested a possibility that carnosine suppresses such decrease of the GABA amount.

Blood was collected from mice starved overnight, and blood insulin concentration was determined with a kit (Morinaga). As a result of the blood test, it was found that increase of the blood insulin concentration observed for the high fat diet-induced Alzheimer disease was suppressed in the carnosine administration group (FIG. 8-4).

### Evaluation 2 with Healthy Volunteers

The same test diet containing chicken-derived imidazole dipeptides as mentioned above was given to test subjects (healthy volunteers 40 years old or older, divided into test diet group and placebo diet group) over three months, and before, during, and after the ingestion period, change of cerebral functions etc. were evaluated. Compositions of the test diet and placebo diet (as daily amounts) are as shown in Table 1. A first pilot test and second pilot test were conducted.

The total numbers of the test subjects subjected to the first and second test were 30 subjects for the test diet group and 30 subjects for the control diet group (placebo diet group) (refer to the following tables).

1. Analysis by MRI imaging

In the second pilot test, MRI imaging test was performed to directly measure the cerebral blood flow, which changes with advance of dementia.

Change of cerebral blood flow can be measured by the arterial spin labeling method, which is a method of measuring blood flow change by using an MRI apparatus using magnetism without using a labeling compound, or the like.

The results are shown in FIGS. 9 and 10. It was found that blood flow in the posterior cingulated gyrus, where blood flow changes with the advance and onset from a predemential stage, was maintained in the test diet ingestion group with a significant difference (p<0.005) compared with the placebo diet group.

### 2. Subgroup Analysis

For the subgroups of the test subjects (60 years old or older) who participated in the first and second pilot tests, logical memory, which degrades with advance and onset from a predemential stage, was evaluated (delayed word recall task).

The results are shown in FIGS. 11 and 12. In this test, difficulty of the second test was higher than that of the first test, and therefore the scores tended to worsen in the second test compared with first test. However, it was found that the degradation of the scores was suppressed in the test diet ingestion group compared with the placebo diet group with a strong statistical significance (p<0.01).

The results of these two experiments suggest that the agent containing the imidazole dipeptides derived from chicken has an action of preventing aging of cerebral functions and onset of dementia.

### Preparation Examples

### (1) Preparation of Chicken Extract

The test diet containing chicken-derived imidazole dipeptides was prepared according to the following process.

Chicken breast meat was minced with a meat grinder, and warm water was added to the chicken breast meat in a weight of 1.5 times the weight of the meat. The mixture was heated at 90° C. for 4 hours, thereby concentrated until Brix became 20% or higher, and subjected to diatomaceous earth filtration and ultrafiltration so that it finally had a carnosine+anserine concentration of about 10% (w/v %).

### (2) Capsules

Carnosine (1.0 weight part), placental extract (powder, 0.2 weight part), and lactose (1.3 weight parts were mixed into a uniform mixture, and filled into hard capsules in a conventional manner to prepare 250 mg net weight capsules (100 mg of carnosine/capsule).

### (3) Tablets

There were prepared tablets containing 60 mg of a mixture of carnosine and anserine per one tablet (300 mg) together with maltose, dextrin, starch, vitamin E-containing vegetable oil, isomaltooligosaccharides, hardly digestible dextrin, shellfish calcium, trehalose, sucrose ester, vitamin C, citric acid, calcium phosphate, perfume, shellac, niacin, vitamin K, sweetener, potassium chloride, vitamin A, calcium pantothenate, biotin, iron pyrophosphate, B vitamins, vitamin D, magnesium carbonate, and folic acid.

### Sequence Listing Free Text

SEQ ID NO: 1—SLC23A2, NM_203327
SEQ ID NO: 2—SLC43A2, NM_152346
SEQ ED NO: 3—SLC29A3, NM_018344
SEQ ID NO: 4—SLC35C1, NM_018389
SEQ ID NO: 5—SLC25A33, NM_032315
SEQ ID NO: 6—SLC22A23, NM_015482
SEQ ID NO: 7—CXCL12, NM_199168
SEQ ID NO: 8—CCL17, NM_002987
SEQ ID NO: 9—TSPO NM_000714
SEQ ID NO: 10—P2RY1, NM_002563
SEQ ID NO: 11—CAMK1, NM_003656
SEQ ID NO: 12—ACO2, NM_001098
SEQ ID NO: 13—ATP7A, NM_000052
SEQ ID NO: 14—POLG, NM_002693
SEQ ID NO: 15—IDH3G, NM_004135
SEQ ID NO: 16—UCP2, NM_003355
SEQ ID NO: 17—BCKDHA, NM_000709
SEQ ID NO: 18—TAP2, NM_018833
SEQ ID NO: 19—SMARCD1, NM_139071
SEQ ID NO: 20—SIRT6 and NM_016539

1. An agent for improving a neuropsychologic function, which contains at least one selected from the group consisting of an imidazole dipeptide and a metabolite thereof.

2. The agent according to claim 1, wherein the neuropsychologic function is one relevant to the Alzheimer disease or aging.

3. An anti-aging agent based on improvement of a neuropsychologic function, which contains at least one selected from the group consisting of an imidazole dipeptide and a metabolite thereof.

4. An agent for changing expression of at least one kind of gene selected from the group consisting of:
transporter genes SLC23A2, SLC43A2, SLC29A3, SLC35C1, SLC25A33, SLC25A23, SLC6A12, and SLC6A13;
chemokine genes CXCL12 and CCL17;
aging-related genes TSPO and P2RY1;
nervous system gene CAMK1;
mitochondrial genes ACO2, ATP7A, POLG, IDH3G, UCP2, BCKDHA, and TAP2; as well as anti-aging genes SMARCD1 and SIRT6,
which contains at least one selected from the group consisting of an imidazole dipeptide and a metabolite thereof.

5. The agent according to claim 1, which has an anti-inflammatory activity.

6. An agent for controlling blood concentration of at least one kind of cytokine selected from the group consisting of IP-10 (CXCL10), IL-2, IL-5, IL-7, IL-8 (CXCL8), IL-13, G-CSF, and MCP-1 (CCL2), which contains at least one selected from the group consisting of an imidazole dipeptide and a metabolite thereof.

7. The agent according to claim 1, which has a blood sugar level increase-suppressing action, or blood sugar level-decreasing action.

8. The agent according to claim 1, which has a blood insulin concentration increase-suppressing action, or blood insulin concentration-decreasing action.

3. The agent according to claim 1, which is for ingestion of 200 mg or more as a daily dose of at least one selected from the group consisting of an imidazole dipeptide and a metabolite thereof.

10. A nutritional composition containing 200 mg or more as a daily dose of at least one kind of imidazole dipeptide derived from animal meat.

11. The nutritional composition according to claim 10, wherein at least one kind of the imidazole dipeptide is derived from chicken.

12. The agent or nutritional composition according to claim 1, which further contains creatine and a nucleic acid.

13. The agent or nutritional composition according to claim 1, which is for an elderly person or a person with a slight mood disorder.

14. An agent for a treatment of diabetes and/or Alzheimer disease, which contains at least one selected from the group consisting of an imidazole dipeptide and a metabolite thereof.

15. A dieting method comprising making a subject for whom improvement of a neuropsychologic function is desirable ingest a nutritional composition containing at least one selected from the group consisting of an imidazole dipeptide and a metabolite thereof.

16. A method for determining a neuropsychologic function of a subject, which is based on a carnosine decomposition enzyme (CNDP1) activity of the subject.

17. A method for predicting a neuropsychologic function-improving effect brought by administering to a subject the agent or nutritional composition according to claim 1, which is based on a CNDP1 activity of the subject.

18. A method for detecting improvement or degradation of a neuropsychologic function, which is based on analysis of expression of at least one kind of gene selected from the group consisting of:
transporter genes SLC23A2, SLC43A2, SLC29A3, SLC35C1, SLC25A33, SLC25A23, SLC6A12, and SLC6A13,
chemokine genes CXCL12 and CCL17;
aging-related genes TSPO and P2RY1;
nervous system gene CAMK1;
mitochondrial genes ACO2, ATP7A, POLG, IDH3G, UCP2, BCKDHA, and TAP2; as well as anti-aging genes SMARCD1 and SIRT6.

19. A kit for detecting improvement or degradation of a neuropsychologic function which comprises
a nucleic acid comprising at least one kind of nucleotide sequence selected from an entire or partial sequence of any one of the nucleotide sequences of SEQ ID NOS: 1 to 20, and
an entire or partial sequence of a nucleotide sequence complementary to any one the nucleotide sequences of SEQ ID NOS: 1 to 20.

20. A pharmaceutical composition for a treatment of aging of cerebral function and/or dementia, which contains a compound represented by the formula I or II: 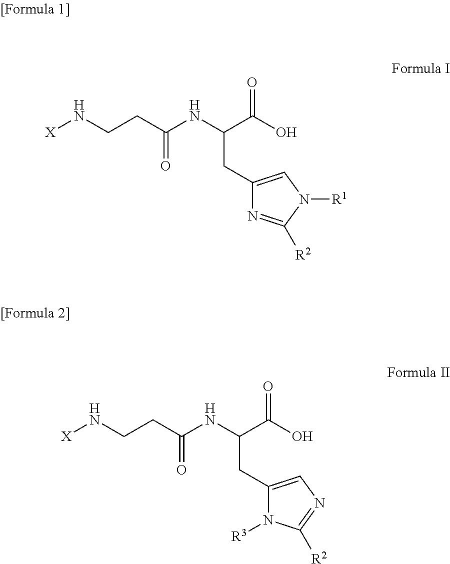
wherein, in the formulas I and II, R¹, R², and R³ are independently H or a C₁₋₆ and X is H or —COR⁴, wherein R⁴ is H or a C₁₋₆ alkyl.

21. A method for searching for an active ingredient for a treatment of aging of a cerebral function and/or dementia, which uses a compound represented by the formula I or II defined in claim 20 as a leading compound.
